(19) United States
(12) Patent Application Publication
(10) Pub. No.: US 2026/0242489 A1
(43) Pub. Date: Aug. 20, 2026

(54) PH-DEPENDENT ANTI-EGFR ANTIBODIES FOR TREATMENT OF SOLID TUMORS

(71) Applicant: HUAHUI HEALTH LTD., Beijing (CN)

(72) Inventors: Jianhua SUI, Beijing (CN); Ximing LIU, Beijing (CN); Xinxin TIAN, Beijing (CN)

(21) Appl. No.: 19/160,100

(22) PCT Filed: Nov. 20, 2023
(86) PCT No.: PCT/CN2023/132753

§ 371 Date: Aug. 27, 2025

(30) Foreign Application Priority Data
Nov. 21, 2022 (WO) PCT/CN2022/133341

(51) Int. Cl. C07K 16/28

(52) U.S. Cl. CPC C07K 16/2863; C07K 2317/21; C07K 2317/622; C07K 2317/92

(57) ABSTRACT

The present disclosure provides monoclonal anti-human EGFR (hEGFR) antibodies with cross-reactivity to human EGFR and mouse EGFR, pH dependent binding and/or competitive binding to hEGFR with its ligand EGF. The present disclosure also provides a method of site-directed engineering of an antibody to increase the pH-dependent binding to antigen of said antibody.

### FIELD OF THE INVENTION

The present disclosure provides monoclonal antibodies, particularly monoclonal antibodies that specifically bind to human EGFR, and preferably specifically binds to both human EGFR and mouse EGFR. Also provided are nucleic acid molecules encoding the anti-EGFR antibodies, as well as expression vectors and host cells comprising the nucleic acid molecules. Pharmaceutical compositions, conjugates and multi-specific antibodies comprising the anti-EGFR antibodies are also disclosed. Also provided are methods for treating various diseases, in particular tumors, e.g. EGFR-expressing tumors.

### BACKGROUND

The human epidermal growth factor receptor (hEGFR) is a transmembrane glycoprotein with an amino-terminal 621 amino acid (Leu25-Ser645) extracellular region (ECR) comprising 4 domains (I-IV). EGFR can be activated by at least seven different growth factors (EGF, transforming growth factor-a (TGFa), betacellulin (BTC), heparin binding EGF-like growth factor (HB-EGF), epiregulin (EREG), epigen (EPGN), and amphiregulin (AREG)) with varying receptor-binding affinities, to regulate cell proliferation and differentiation. EGF is a high-affinity ligand of EGFR, it binds with EGFR domains I and III to stabilize EGFR dimerization, which promotes activation of receptor tyrosine kinases (RTKs). EGFR plays a causal role in the development and maintenance of many types of cancer and is a well-validated oncology target.

Therapeutic anti-EGFR antibodies including Cetuximab, Panitumumab, and Necitumumab, which bind to domain III of human EGFR, are in use for the clinical treatment of solid tumors including colorectal cancer and non-small-cell lung cancer (NSCLC) (Cai, W. Q. et al., 2020). In addition, many investigations based on anti-EGFR antibodies (and related antibody-based therapies) for cancer therapy are ongoing.

However, given that EGFR is expressed in normal tissues of diverse origins (e.g., epithelial, mesenchymal and neuronal), and considering that EGFR is known to affect normal cellular processes including proliferation, differentiation, and development, there are problems with on-target/off-tumor toxicity that have limited the clinical utility of anti-EGFR antibodies (Hansel, T. T. et al., 2010; Achermann, Y. et al., 2012).

Nimotuzumab is another therapeutic anti-EGFR antibody with ~10 fold lower binding affinity than Cetuximab (Crombet Ramos, T. et al., 2020). In contrast to Cetuximab's binding to normal cells with low EGFR expression levels, Nimotuzumab selectively binds with tumor cells with high EGFR levels, so it follows that Nimotuzumab treatment exhibits relatively low on-target off-tumor toxicity and is suitable for patients with high EGFR expression or gene amplification (Crombet Ramos, T. et al., 2020; Garrido, G. et al., 2011).

EGFRvIII is a common ECR truncation mutant of EGFR found with glioblastoma multiforme (GBM), head and neck cancer, and breast cancer; it has an in-frame deletion of 267 amino acids from EGFR ECR domains I and II (Gan, H. K. et al., 2012). EGFRvIII is tumor specific, and there is no evidence that it occurs in normal tissues. Illustrating the identification of a tumor selective anti-EGFR antibody (DH8.3) by targeting tumor-specific antigens, an antibody with higher binding affinity for EGFRvIII than wild type (WT) EGFR showed tumor selectivity in glioblastoma expressing EGFRvIII (Jungbluth, A. A. et al., 2003; Hills, D., Rowlinson-Busza, G., and Gullick, W. J., 1995); note that antibody specific targeting EGFRvIII cannot exhibit tumor selectivity in cancer types expressing WT EGFR but not EGFRvIII (e.g. colorectal cancer and pancreatic cancer) (Gan, H. K., Burgess, et al., 2012).

Therefore, there's an ument need of tumor-selective anti-EGFR antibodies with desirable binding affinity.

Mouse tumor models are widely used during the preclinical development of antitumor therapies. While many anti-EGFR antibodies can elicit potent antitumor activity in preclinical mouse tumor models, most therapeutic anti-EGFR antibodies can recognize human EGFR but not mouse EGFR (mEGFR); thus, the on-target/off-tumor effects and other problems resulting from antibody binding with EGFR in normal tissues cannot be predicted based on experimental programs using mouse tumor models. Humanized mice provide a solution for evaluating the performance of therapeutic antibodies in vivo. However, EGFR humanized mice are difficult to obtain, owing to the essential function of mouse EGFR binding with its diverse ligands, and EGFR deficiency is known to lead to multiple abnormalities in mice (Roskoski, R., Jr., 2014; Threadgill, D. W. et al., 1995). Thus, developing anti-EGFR antibodies with adequate cross-reactive binding with hEGFR and mEGFR should greatly aid evaluations (e.g., pharmacokinetics, safety, and therapeutic efficacy) of anti-EGFR antibodies in mouse tumor models.

### SUMMARY OF THE INVENTION

The present inventors have developed pH-dependent anti-EGFR antibodies to overcome limitations with anti-EGFR antibodies owing to EGFR expression in normal tissues. The inventors initially generated antibodies with cross-species reactivity for human and mouse EGFR by screening a non-immune human antibody library and through antibody affinity maturation. The inventors then generated a structure model of an anti-EGFR antibody in complex with EGFR, and interrogated it for residues affecting pH-dependent binding, which supported development of a cross-reactive pH-dependent anti-EGFR antibody, G532, as well as a non-pH-dependent variant of G532 (G532^{Ctrl}). The inventors experimentally confirmed that the pH-dependent binding of G532 improves its tumor selectivity, tumor penetration, and antitumor activity over G532^{Ctrl}.

In a first aspect, the present disclosure relates to isolated antibody or an antigen-binding fragment thereof that specifically binds to human EGFR (hEGFR), and has one or more of following properties: cross-reactivity to mouse EGFR, pH-dependent binding and/or competitive binding to hEGFR with its ligand EGF.

In a second aspect, the present disclosure relates to an isolated polynucleotide encoding the antibody or fragment thereof of the first aspect.

In a third aspect, the present disclosure relates to an expression vector comprising the isolated polynucleotide of the second aspect.

In a fourth aspect, the present disclosure relates to a host cell comprising the isolated polynucleotide of the second aspect or the expression vector of the third aspect.

In a fifth aspect, the present disclosure relates to a composition, e.g., a pharmaceutical composition, comprising the antibody or fragment thereof of the first aspect, and a pharmaceutically acceptable carrier. Preferably, the pharmaceutical composition comprises a therapeutically effective amount of the antibody or fragment thereof of the first aspect.

In a six aspect, the present disclosure provides a method of reducing tumors, inhibiting the growth of tumor cells, treating a cancer or preventing recurrence of a cancer in a subject in need thereof, comprising administering to the subject a therapeutically effective amount of the anti-EGFR antibody or fragment thereof of the first aspect or the composition of the fifth aspect.

In a seventh aspect, the present disclosure provides use of the anti-EGFR antibody or fragment thereof of the first aspect in the manufacture of a medicament. In one embodiment, the medicament is used for reducing tumors, inhibiting the growth of tumor cells, treating a cancer, preventing recurrence of cancer.

The present application also practically demonstrates a structure-guided antibody-antigen binding pH-dependency engineering strategy that can be applied for developing pH-dependent antibodies targeting other tumor-associated antigens that are expressed in normal tissues.

Accordingly, in an eighth aspect, the present disclosure relates to a site-directed engineering method of preparing a pH-dependent antibody targeting an antigen. Specifically, the method comprises:
(i) providing an antibody that specifically binds to an antigen;
(ii) identifying the amino acids contributing to the antigen-antibody binding, wherein one or more of said amino acids contributing to the antigen-antibody binding is histidine;
(iii) identifying the amino acids on the antibody that interact with the histidines as identified in step (ii), and replacing one or more of the amino acids on the antibody with acidic amino acid to obtain one or more variant of said antibody; and
(iv) determining the pH-dependent binding of the variants of said antibody obtained in step (iii) to identify the variant antibody with increased pH-dependent binding.

### BRIEF DESCRIPTION OF THE DRAWINGS

FIG. 1 is a schematic diagram showing the process of screening pH-dependent antibodies from a non-immune phage display human scFv antibody library.

FIG. 2 shows the binding of 29 phage display scFv antibodies to hEGFR ECR at pH 6.0 and at pH 7.4, analyzed with ELISA.

FIGS. 3A-B show the comparison of binding activity of the pH-dependent anti-human EGFR antibodies 11A10 and 14C07 with Cetuximab. (A) Cetuximab, 11A10, and 14C07 binding with hEGFR ECR at pH 6.0, and pH 7.4, analyzed with ELISA. (B) Ligand-binding blocking activity of Cetuximab, 11A10, and 14C07 for EGF binding to hEGFR at pH 6.0, analyzed with ELISA. The IC₅₀ values are shown. n.m. (not measurable).

FIG. 4 shows binding of Cetuximab and 14C07 (in human IgG1 form) with WT hEGFR ECR, and its two variants containing H370A or H433A mutation at pH 6.5, and pH 7.4. The EC₅₀ values and binding pH-dependency (EC₅₀ ratio (pH 7.4/pH 6.5)) is shown in the table. n.m.=not measurable.

FIG. 5A-B show binding cross-reactivity of the antibodies to human and mouse EGFR. (A) Binding of 14C07 and G5V2 to human and mouse EGFR at pH 6.5 and pH 7.4. The EC₅₀ values and binding pH-dependency (EC₅₀ ratio (pH 7.4/pH 6.5)) are shown in the table. (B) Binding of Cetuximab to human and mouse EGFR at pH 7.4.

FIG. 6 shows the binding activity of G5V2 and Cetuximab to human EGFR ECD and human EGFR domain III.

FIG. 7 shows a complex structure model of G5V2-hEGFR. Carbon atoms are colored in cyan for the VL of G5V2, green for the VH of G5V2, and gray for the hEGFR ECR. Ribbon representation of G5V2 scFv and hEGFR ECR in orthogonal views (left). A detailed view of the G5V2-hEGFR interface revealing the locations of G5V2 CDRs and hEGFR residues H370, H433, R377, L406, Q435, and K489 (right).

FIG. 8 shows a detailed view of the G5V2-hEGFR complex model revealing the locations of amino acids on hEGFR domain III that are distinct from mouse EGFR (colored in orange).

FIG. 9 shows the G5V2-hEGFR complex model indicating that the G5V2 LCDR1 Y32 residue is positioned in close proximity with hEGFR H433, and that the G5V2 LCDR2 D52D53 residues face hEGFR H370.

FIGS. 10A-B show binding of G5V2 to hEGFR ECR and its variants. (A) G5V2 binding at pH 7.4 with the WT hEGFR ECR and its variants containing multiple mutations (R377A, L406A, Q435A, K489A), analyzed with ELISA. (B) G5V2 binding at pH 7.4 with WT hEGFR ECR, and its two variants containing an H370A or H433A mutation.

FIG. 11 shows binding activity of the phage display Fab form of WT G5V2, and its variants (G5V2 Y32E, G5V2 Y32D, G5V2 Y32H) to hEGFR ECR at pH 6.5 and pH 7.4. The EC₅₀ values, and binding pH-dependency (EC₅₀ ratio (pH 7.4/pH 6.5)) are shown in the table below. n.m. (not measurable).

FIGS. 12A-B show the generation and characterization of G532 from structure-guided engineering of G5V2. (A) Schematic depicting the process of structure-guided engineering of G5V2. The schematic diagram illustrates relationships between indicated antibodies (left), and sequence alignment of HCDR2, LCDR1, and LCDR2 of the indicated antibodies (right) are shown. (B) G5V2, G532, G532^{V}, and G532^{Ctrl} (in human IgG1 form) binding with hEGFR ECR at pH 6.5 and pH 7.4. The EC₅₀ values and binding pH-dependency (EC₅₀ ratio (pH 7.4/pH 6.5)) are shown in the table at the right.

FIGS. 13A-B shows SPR analysis of binding affinity and pH-dependency of G532 and of G532^{Ctrl} with hEGFR and mEGFR. (A) hEGFR. (B) mEGFR. The dissociation constant (KD) values were calculated with a steady-state affinity model using the Biacore T200 valuation software, and are shown in Table 2.

FIG. 14 shows FACS analysis of human EGFR expression in WT CT26 or CT26-hEGFR cells with Cetuximab.

FIG. 15A-B shows FACS analysis of G532 and G532^{Ctrl} (in human IgG1 form) binding with A-431 cells (A), and CT26-hEGFR cells (B) at pH 6.5 and pH 7.4. Representative histograms showing antibody-cell binding at the antibody concentration of 0.823 nM (left); binding curves of median fluorescence intensity (MFI) vs. antibody concentration (right) are shown.

FIG. 16 shows ligand-binding blocking activity of Control IgG, G532, and G532^{Ctrl} (in human IgG1 form) for EGF binding to hEGFR at pH 6.5 and pH 7.4. The IC₅₀ values are shown in the table below n.m. (not measurable).

FIG. 17 shows immunofluorescence images of whole-tumor specimens showing penetration of Control IgG, G532, or G532^{Ctrl} into A-431 tumors. Each images use its individual scar bar.

FIG. 18 shows representative immunofluorescence images (above), and their human IgG immunohistochemistry images (below) converted by Inform software. Scale bar, 100 μm.

FIG. 19 shows integrated density of FTIC in mice livers, and A-431 tumors analyzed with ImageJ. Data are means±s.e.; statistical significance was determined using Two-tailed unpaired Student's t-tests.

FIG. 20 shows integrated density ratio of FITC in A-431 tumors and related mice livers. Data are means±s.e.; statistical significance was determined using Two-tailed unpaired Student's t-tests.

FIG. 21 shows representative human IgG immunohistochemistry images of all mice livers and A-431 tumors.

FIG. 22A-C show Characterization of antitumor activity of G532. (A) Antitumor activity of G532 and G532^{Ctrl} in A-431 tumor models. CB-17 SCID mice bearing A-431 tumors were randomized into three groups with similar tumor volumes: Control IgG, G532, or G532^{Ctrl} (10 mg/kg, in human IgG1 form). (B) Antitumor activity of G532, and G532^{Ctrl} in CT-26-hEGFR tumor models. Balb/c mice bearing CT-26-hEGFR tumors were randomized into three groups with similar tumor volumes: Control IgG, G532, or G532^{Ctrl} (10 mg/kg, in human IgG1 form). (C) Antitumor activity of G532, and Cetuximab in A-431 tumor models. CB-17 SCID mice bearing A-431 tumors were randomized into three groups with similar tumor volumes: Control IgG, G532, or Cetuximab (10 mg/kg, in human IgG1 form). (A-C) Antibodies were tested at 10 mg/kg. The time points for antibody treatment are marked by arrows. Data are means±s.e.; statistical significance was determined using Two-way ANOVA.

### DETAILED DESCRIPTION OF THE INVENTION

Unless specifically defined elsewhere in this document, all the technical and scientific terms used herein have the meaning commonly understood by one of ordinary skill in the art to which this invention belongs.

### Definitions

Unless specifically defined elsewhere in this document, all other technical and scientific terms used herein have the meaning commonly understood by one of ordinary skill in the art to which this invention belongs.

Although any methods and materials similar or equivalent to those described herein may be used in the practice for testing of the present disclosure, exemplary materials and methods are described herein. In describing and claiming the present disclosure, the following terminology will be used.

As used herein, including the appended claims, the singular forms of words such as “a”, “an”, and “the”, include their corresponding plural references unless the context clearly dictates otherwise.

The term “or” is used to mean, and is used interchangeably with, the term “and/or” unless the context clearly dictates otherwise.

In the context of the present disclosure, unless being otherwise indicated, the wording “comprise”, and variations thereof such as “comprises” and “comprising” will be understood to imply the inclusion of a stated element, e.g. an amino acid sequence, a nucleotide sequence, a property, a step or a group thereof, but not the exclusion of any other elements, e.g. amino acid sequences, nucleotide sequences, properties and steps. When used herein the term “comprise” or any variation thereof can be substituted with the term “contain”, “include” or sometimes “have” or equivalent variation thereof. In certain embodiments, the wording “comprise” also includes the scenario of “consisting of”.

The term “antibodies” herein should be understood in a broad sense and includes immunoglobulin molecules including monoclonal antibodies, antibody fragments, multispecific antibodies, dimeric, tetrameric, or multimeric antibodies, single chain antibodies, domain antibodies and any other modified configuration of the immunoglobulin molecule that comprises an antigen binding site of the required specificity. The most commonly seen basic structure of an antibody is a tetramer. Each tetramer includes two identical pairs of polypeptide chains, each pair having one smaller chain designated as the “light chain” and one bigger chain designated as the “heavy chain”. At the amino-terminus of each chain, it includes a variable domain of about 100 to 110 or more amino acids primarily responsible for antigen recognition. The carboxy-terminal portion of the heavy chain may define a constant region primarily responsible for effector function.

The term “hypervariable domain” means the amino acid residues of an antibody that are responsible for antigen-binding. The hypervariable domain comprises amino acid residues from a “complementarity determining region (CDR)” (i.e., LCDR1, LCDR2 and LCDR3 in the light chain variable domain and HCDR1, HCDR2 and HCDR3 in the heavy chain variable domain). Typically, the variable domains of both the heavy and light chains comprise three hypervariable domains, namely CDRs, which are located between relatively conserved “framework regions” or “FR”. The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). Kabat numbering system is used for the amino acid residues of the antibody unless indicated otherwise (Kabat et al., Sequence of proteins of immunological interest, 5th ed., Public Health Service, National Institute of Health, Bethesda, MD (1991)).

By “specific binding” or “specifically bind to”, it means that an antibody exhibits preferential binding to a certain target as compared to other proteins, but this specificity does not require absolute binding specificity. An antibody is considered “specific” for its intended target if its binding is determinative of the presence of the target protein in a sample, e.g. without producing undesired results such as false positives. The antibody or an antigen binding fragment thereof of the present invention will bind to the target protein with an affinity that is at least 2-fold greater, preferably at least 10-fold greater, more preferably at least 20-fold greater, and most preferably at least 100-fold greater than the affinity with non-target proteins.

“Anti-EGFR” refers to an antibody or antibody domain that can bind to EGFR, specifically human EGFR having an amino acid sequence as shown in SEQ ID NO: 1. The style “EGFR” refers to the EGFR protein or EGFR gene product. The style “EGFR” refers to the EGFR gene.

By “pH-dependent binding” of an antibody, it means that the antibody binds to its antigen at different affinity under different pH. In particular, the pH-dependent binding of an antibody of the present application means that the antibody binds to EGFR, e.g. hEGFR, or both hEGFR and mEGFR with a higher affinity under mild acidic condition (e.g. at a pH of tumor microenvironment) as compared to its binding to the same target under neutral pH (e.g. at a pH of normal tissues).

By “cross-reactive” of an antibody in the context of the present disclosure, it specifically refers to ability to react with or bind to homologous or orthologous targets across different species. More specifically, the “cross-reactivity” means that an antibody can binds to both human and mouse target, specifically EGFR. The cross-reactivity of an antibody can be determined using any method as known in the art. For example, it can be determined by measurement of binding affinity via surface plasmon resonance (e.g., BIACORE) or a similar technique (e.g. KinExa or OCTET).

The term “monoclonal antibody” or “mAb” or “Mab” as used herein refers to a population of substantially homogeneous antibodies, which means that the antibody molecules comprised in the population are identical in amino acid sequence except for possible naturally occurring mutations that may be present in minor amounts. By “monoclonal”, it indicates the character of the antibody as being obtained from a substantially homogeneous population of antibodies, and is not to be construed as requiring the antibody to be produced by any particular method. Monoclonal antibodies (mAbs) may be obtained by conventional methods known in the art.

The antibody or the antigen-binding fragment of the present invention can be an isolated antibody. By the term “isolated” it means that the antibodies or antigen-binding fragments are at least partially free of other biological materials or non-biological materials from the cells, cell cultures, growth medium, expression system in which they are produced. Said materials may include nucleic acids, proteins, lipids, carbohydrates, buffer, salt or other material such as cellular debris and growth medium.

The present application also provides an isolated nucleic acid sequence comprising a nucleotide sequence coding for the antibody or fragment thereof the present application. By “isolated nucleic acid” or “isolated polynucleotide”, it means a DNA or RNA which is removed from all or a portion of a polynucleotide in which the isolated polynucleotide is found in nature, or is linked to a polynucleotide to which it is not linked in nature. An isolated nucleic acid molecule “comprising” a specific nucleotide sequence may include, in addition to the specified sequence, operably linked regulatory sequences that control expression of the coding region of the recited nucleic acid sequences. Due to the codon degeneracy, one skilled in the art can understand that a specific amino acid sequence can be coded by different nucleotide sequences.

The terms “administration”, “administering”, “treating” and “treatment” as used herein, when applied to a subject, e.g. an animal, including human, or to cells, tissue, organ, or biological fluid, mean contact of an exogenous pharmaceutical, therapeutic, diagnostic agent, or composition to the subject, cell, tissue, organ, or biological fluid. Treatment of a cell encompasses contact of a reagent to the cell, as well as contact of a reagent to a fluid, where the fluid is in contact with the cell. The term “administration” and “treatment” also include in vitro and ex vivo treatments, e.g., of a cell, by a reagent, diagnostic, binding compound, or by another cell.

The term “therapeutically effective amount” as used herein, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated.

In the context of the present application, by “subject” it refers to an animal, preferably a mammal, e.g., a primate, preferably a higher primate, e.g., a human.

The terms “cancer” or “tumor” herein mean or describe the physiological condition in mammals that is typically characterized by unregulated cell growth.

Structure-Guided Engineering to Generate Antibodies with pH-Dependent Binding

Histidine (H) has been repeatedly demonstrated to contribute to the binding between pH-dependent antibodies and their antigens (Johnston, R. J. et al., 2019; Kang, J. C. et al., 2019). Based on this finding, previous engineering method to generate antibodies with pH-dependent binding comprises generating a library of antibody variants by substituting each of the amino acids in CDRs with histidine (H), and screening the library to identify engineered antibodies with improved pH-dependent binding. Such process is time-consuming and labor intensive, as it requires extensive screening of variants.

In the present application, the inventors developed a site-directed engineering process. Instead of histidine substitution of CDR amino acids, the inventors intend to establish interaction between histidine and acidic acid by identifying the histidine on antigen that interacts with antibody and contributes to the antigen-antibody binding, and replacing the antibody amino acid that interacts with said histidine on the antigen.

Therefore, the present application provides a method of engineering an antibody to increase the pH-dependent binding of said antibody, comprising:
(i) providing an antibody that specifically binds to an antigen;
(ii) identifying the amino acids contributing to the antigen-antibody binding, wherein one or more of said amino acids contributing to the antigen-antibody binding is histidine;
(iii) identifying the amino acids on the antibody that interact with the histidines as identified in step (ii), and replacing one or more of the amino acids on the antibody with acidic amino acid to obtain one or more variant of said antibody; and
(iv) determining the pH-dependent binding of the variants of said antibody obtained in step (iii) to identify the variant antibody with increased pH-dependent binding.

The antibody provided in step (i) can either be an antibody with or without pH-dependent binding specificity.

In step (ii), the identification of the histidine on antigen can be conducted based on 3D model of antigen-antibody complex. For example, the identification of histidine contributing to antigen-antibody binding may comprise selecting one or more histidines in close proximity to one or more amino acids on the antibody, and further confirming the contribution of histidine to antigen-antibody binding by substituting it with a different amino acid, e.g. alanine. If the antigen-antibody binding is significantly reduced after the substitution, the histidine can be identified as one contributing to antigen-antibody binding. For example, the histidine on antigen that contributes to antigen-antibody binding can be a part of the epitope.

In step (iii), the identification of antibody amino acids that interacts with the histidine can also be conducted based on 3D antigen-antibody complex model. The acidic amino acid of step (iii) can be glutamate (E) or aspartate (D).

In step (iv), the variant antibodies obtained by substitution can be tested for its binding properties at different pH to determine its pH-dependent binding. For example, binding properties, such as binding affinity and specificity can be tested at an acidic pH, e.g. pH 6.0 or pH 6.5 and a neutral pH, e.g. pH 7.4.

### Anti-EGFR Antibodies

The present inventors, by screening a non-immune human antibody library and the novel site-directed engineering method of the present application, developed a group of isolated antibodies that specifically bind to human EGFR (hEGFR), and have one or more characteristic properties including cross-reactivity to mouse EGFR, pH-dependent binding to antigen, and/or competitive binding to hEGFR with its ligand EGF. Preferably, the anti-hEGFR antibody of the present application has two or three of said characteristic properties.

In some embodiments, the anti-hEGFR antibody of the present application specifically binds to human EGFR (hEGFR), which comprises any one of following (a) to (h):
(a) HCDR1, HCDR2 and HCDR3 comprising or consisting of an amino acid sequence of SEQ ID NOs: 50, 61 and 52, respectively; and LCDR1, LCDR2, LCDR3 comprising or consisting of an amino acid sequence of SEQ ID NOs: 62, 15 and 54 (G532);
(b) HCDR1, HCDR2 and HCDR3 comprising or consisting of an amino acid sequence of SEQ ID NOs: 11, 12 and 13, respectively; and LCDR1, LCDR2, LCDR3 comprising or consisting of an amino acid sequence of SEQ ID NOs: 14, 15 and 16 (14C07);
(c) HCDR1, HCDR2 and HCDR3 comprising or consisting of an amino acid sequence of SEQ ID NOs: 50, 51 and 52, respectively; and LCDR1, LCDR2, LCDR3 comprising or consisting of an amino acid sequence of SEQ ID NOs: 53, 15 and 54 (G5V2);
(d) HCDR1, HCDR2 and HCDR3 comprising or consisting of an amino acid sequence of SEQ ID NOs: 50, 61 and 52, respectively; and LCDR1, LCDR2, LCDR3 comprising or consisting of an amino acid sequence of SEQ ID NOs: 69, 70 and 54 (G532^{V});
(e) HCDR1, HCDR2 and HCDR3 comprising or consisting of an amino acid sequence of SEQ ID NOs: 50, 61 and 52, respectively; and LCDR1, LCDR2, LCDR3 comprising or consisting of an amino acid sequence of SEQ ID NOs: 77, 70 and 54 (G532^{Ctrl});
(f) HCDR1, HCDR2 and HCDR3 comprising or consisting of an amino acid sequence of SEQ ID NOs: 23, 24, and 25 respectively; and LCDR1, LCDR2, LCDR3 comprising or consisting of an amino acid sequence of SEQ ID NOs: 26, 27, and 28 (11A10);
(g) HCDR1, HCDR2 and HCDR3 comprising or consisting of an amino acid sequence of SEQ ID NOs: 31, 32, and 33, respectively; and LCDR1, LCDR2, LCDR3 comprising or consisting of an amino acid sequence of SEQ ID NOs: 34, 35, and 36 (11C04); or
(h) HCDR1, HCDR2 and HCDR3 comprising or consisting of an amino acid sequence of SEQ ID NOs: 11, 39 and 40, respectively; and LCDR1, LCDR2, LCDR3 comprising or consisting of an amino acid sequence of SEQ ID NOs: 41, 42, and 43 (11C05); wherein the hEGFR has an amino acid sequence as shown in SEQ ID NO: 1.

In preferred embodiment, the anti-hEGFR antibody or fragment thereof shows cross-reactivity to mouse EGFR (SEQ ID NO: 3). Specifically, the anti-hEGFR antibody or fragment thereof shows binding affinity to both human and mouse EGFRs, as represented by a KD value of lower than 1×10⁻⁶ M, lower than 1×10⁻⁷ M, lower than 1×10⁻⁸ M, or lower than 1×10⁻⁹ M (1 nM).

In preferred embodiment, the anti-hEGFR antibody or fragment thereof has pH dependence in binding to its target, human EGFR, or preferably both human and mouse EGFR, wherein the binding activity of the antibody or fragment thereof to its target at acidic pH is higher than the binding activity at neutral pH. For example, the binding activity at acidic pH is at least 2-fold, 3-fold, 5-fold, 10-fold, 15-fold, 20-fold, 30-fold, 40-fold, 50-fold, 60-fold, 70-fold, 80-fold, 90-fold, or 100-fold higher as compared to the binding activity at neutral pH. The binding activity can be measured by any method known in the art, e.g. ELISA, FACS, or surface plasmon resonance (e.g. Biacore®). For example, when the binding activity to hEGFR or mEGFR is measured by ELISA assay, the EC50 of the antibody or fragment thereof at acidic pH is at least 2-fold, 3-fold, 5-fold, 10-fold, 15-fold, 20-fold, 30-fold, 40-fold, 50-fold, 60-fold, 70-fold, 80-fold, 90-fold, or 100-fold lower than the EC50 at neutral pH. The acidic pH is 5.8, 5.9, 6.0, 6.1, 6.2, 6.3, 6.4, 6.5, 6.6, 6.7 or 6.8, specifically 6.5. The neutral pH is 7.0, 7.1, 7.2, 7.3, 7.4, 7.5 or 7.6, specifically 7.4. In one embodiment, the antibody or fragment thereof has desirable binding activity at acidic pH, and has no binding activity or considerably low binding activity at neutral pH.

In preferred embodiments, the anti-hEGFR antibody or fragment thereof blocks the binding of human EGFR to its ligand human EGF. For example, the binding of EGFR to EGF is reduced by the antibody of the present application by at least 10%, at least 25%, at least 50%, at least 60%, at least 70%, at least 80%, at least 90%.

In one embodiment, the anti-hEGFR antibody of the present application comprises a VH comprising or consisting of an amino acid sequence of SEQ ID NO: 55 or an amino acid sequence having at least 85%, at least 90%, at least 95%, at least 98% sequence identity to SEQ ID NO: 55, and a VL comprising or consisting of an amino acid sequence of SEQ ID NO: 56 or an amino acid sequence having at least 85%, at least 90%, at least 95%, at least 98% sequence identity to SEQ ID NO: 56. More specifically, the anti-hEGFR antibody comprises a heavy chain comprising or consisting of an amino acid sequence of SEQ ID NO: 57, or an amino acid sequence having at least 85%, at least 90%, at least 95%, at least 98% sequence identity to SEQ ID NO: 57, and a light chain comprising or consisting of an amino acid sequence of SEQ ID NO: 58, or an amino acid sequence having at least 85%, at least 90%, at least 95%, at least 98% sequence identity to SEQ ID NO: 58. In this embodiment, the anti-hEGFR antibody shows cross-reactivity to mouse EGFR, pH-dependent binding to antigen, and competitive binding to hEGFR with its ligand EGF. In a specific embodiment, the anti-hEGFR antibody is G532. G532 is able to bind to both human and mouse EGFRs in a pH-dependent manner. G532 inhibits the binding of hEGFR to its ligand EGF by competitively binding to hEGFR.

In one embodiment, the anti-hEGFR antibody of the present application comprises a VH comprising or consisting of an amino acid sequence of SEQ ID NO: 5 or an amino acid sequence having at least 85%, at least 90%, at least 95%, at least 98% sequence identity to SEQ ID NO: 5, and a VL comprising or consisting of an amino acid sequence of SEQ ID NO: 6 or an amino acid sequence having at least 85%, at least 90%, at least 95%, at least 98% sequence identity to SEQ ID NO: 6. More specifically, the anti-hEGFR antibody comprises a heavy chain comprising or consisting of an amino acid sequence of SEQ ID NO: 7, or an amino acid sequence having at least 85%, at least 90%, at least 95%, at least 98% sequence identity to SEQ ID NO: 7, and a light chain comprising or consisting of an amino acid sequence of SEQ ID NO: 8, or an amino acid sequence having at least 85%, at least 90%, at least 95%, at least 98% sequence identity to SEQ ID NO: 8. In this embodiment, the anti-hEGFR antibody shows cross-reactivity to mouse EGFR, pH-dependent binding to antigen, and competitive binding to hEGFR with its ligand EGF. In a specific embodiment, the anti-hEGFR antibody is 14C07. 14C07 is able to bind to both human and mouse EGFRs in a pH-dependent manner. 14C07 inhibits the binding of hEGFR to its ligand EGF by competitively binding to hEGFR.

In one embodiment, the anti-hEGFR antibody of the present application comprises a VH comprising or consisting of an amino acid sequence of SEQ ID NO: 44 or an amino acid sequence having at least 85%, at least 90%, at least 95%, at least 98% sequence identity to SEQ ID NO: 44, and a VL comprising or consisting of an amino acid sequence of SEQ ID NO: 45 or an amino acid sequence having at least 85%, at least 90%, at least 95%, at least 98% sequence identity to SEQ ID NO: 45. More specifically, the anti-hEGFR antibody comprises a heavy chain comprising or consisting of an amino acid sequence of SEQ ID NO: 46, or an amino acid sequence having at least 85%, at least 90%, at least 95%, at least 98% sequence identity to SEQ ID NO: 46, and a light chain comprising or consisting of an amino acid sequence of SEQ ID NO: 47, or an amino acid sequence having at least 85%, at least 90%, at least 95%, at least 98% sequence identity to SEQ ID NO: 47. In this embodiment, the anti-hEGFR antibody shows cross-reactivity to mouse EGFR. In a specific embodiment, the anti-hEGFR antibody is G5V2. G5V2 is able to bind to both human and mouse EGFRs.

In one embodiment, the anti-hEGFR antibody of the present application comprises a VH comprising or consisting of an amino acid sequence of SEQ ID NO: 63 or an amino acid sequence having at least 85%, at least 90%, at least 95%, at least 98% sequence identity to SEQ ID NO: 63, and a VL comprising or consisting of an amino acid sequence of SEQ ID NO: 64 or an amino acid sequence having at least 85%, at least 90%, at least 95%, at least 98% sequence identity to SEQ ID NO: 64. More specifically, the anti-hEGFR antibody comprises a heavy chain comprising or consisting of an amino acid sequence of SEQ ID NO: 65, or an amino acid sequence having at least 85%, at least 90%, at least 95%, at least 98% sequence identity to SEQ ID NO: 65, and a light chain comprising or consisting of an amino acid sequence of SEQ ID NO: 66, or an amino acid sequence having at least 85%, at least 90%, at least 95%, at least 98% sequence identity to SEQ ID NO: 66. In this embodiment, the anti-hEGFR antibody shows cross-reactivity to mouse EGFR, and pH-dependent binding to antigen. In a specific embodiment, the anti-hEGFR antibody is G532. G532 is able to bind to both human and mouse EGFRs in a pH-dependent manner.

In one embodiment, the anti-hEGFR antibody of the present application comprises a VH comprising or consisting of an amino acid sequence of SEQ ID NO: 71 or an amino acid sequence having at least 85%, at least 90%, at least 95%, at least 98% sequence identity to SEQ ID NO: 71, and a VL comprising or consisting of an amino acid sequence of SEQ ID NO: 72 or an amino acid sequence having at least 85%, at least 90%, at least 95%, at least 98% sequence identity to SEQ ID NO: 72. More specifically, the anti-hEGFR antibody comprises a heavy chain comprising or consisting of an amino acid sequence of SEQ ID NO: 73, or an amino acid sequence having at least 85%, at least 90%, at least 95%, at least 98% sequence identity to SEQ ID NO: 73, and a light chain comprising or consisting of an amino acid sequence of SEQ ID NO: 74, or an amino acid sequence having at least 85%, at least 90%, at least 95%, at least 98% sequence identity to SEQ ID NO: 74. In this embodiment, the anti-hEGFR antibody shows cross-reactivity to mouse EGFR. In a specific embodiment, the anti-hEGFR antibody is G532^{Ctrl}. G532^{Ctrl} is able to bind to both human and mouse EGFRs, while not in a pH-dependent manner.

In one embodiment, the anti-hEGFR antibody of the present application comprises a VH comprising or consisting of an amino acid sequence of SEQ ID NO: 17 or an amino acid sequence having at least 85%, at least 90%, at least 95%, at least 98% sequence identity to SEQ ID NO: 17, and a VL comprising or consisting of an amino acid sequence of SEQ ID NO: 18 or an amino acid sequence having at least 85%, at least 90%, at least 95%, at least 98% sequence identity to SEQ ID NO: 18. More specifically, the anti-hEGFR antibody comprises a heavy chain comprising or consisting of an amino acid sequence of SEQ ID NO: 19, or an amino acid sequence having at least 85%, at least 90%, at least 95%, at least 98% sequence identity to SEQ ID NO: 19, and a light chain comprising or consisting of an amino acid sequence of SEQ ID NO: 20, or an amino acid sequence having at least 85%, at least 90%, at least 95%, at least 98% sequence identity to SEQ ID NO: 20. In a specific embodiment, the anti-hEGFR antibody is 11A10.

In one embodiment, the anti-hEGFR antibody of the present application comprises a VH comprising or consisting of an amino acid sequence of SEQ ID NO: 29 or an amino acid sequence having at least 85%, at least 90%, at least 95%, at least 98% sequence identity to SEQ ID NO: 29, and a VL comprising or consisting of an amino acid sequence of SEQ ID NO: 30 or an amino acid sequence having at least 85%, at least 90%, at least 95%, at least 98% sequence identity to SEQ ID NO: 30. In a specific embodiment, the anti-hEGFR antibody is 11C04.

In one embodiment, the anti-hEGFR antibody of the present application comprises a VH comprising or consisting of an amino acid sequence of SEQ ID NO: 37 or an amino acid sequence having at least 85%, at least 90%, at least 95%, at least 98% sequence identity to SEQ ID NO: 37, and a VL comprising or consisting of an amino acid sequence of SEQ ID NO: 30 or an amino acid sequence having at least 85%, at least 90%, at least 95%, at least 98% sequence identity to SEQ ID NO: 30. In a specific embodiment, the anti-hEGFR antibody is 11C05.

The present application also provides an isolated nucleic acid molecule encoding the antibody of the present application, comprising any one of (a) to (e):
(a) a nucleotide sequence encoding the heavy chain and having at least 95%, 96%, 97%, 98%, 99% or 100% identity to the nucleotide sequence of SEQ ID NO: 59, and/or a nucleotide sequence encoding the light chain and having at least 95%, 96%, 97%, 98%, 99% or 100% identity to the nucleotide sequence of SEQ ID NO: 60;
(b) a nucleotide sequence encoding the heavy chain and having at least 95%, 96%, 97%, 98%, 99% or 100% identity to the nucleotide sequence of SEQ ID NO: 9, and/or a nucleotide sequence encoding the light chain and having at least 95%, 96%, 97%, 98%, 99% or 100% identity to the nucleotide sequence of SEQ ID NO: 10;
(c) a nucleotide sequence encoding the heavy chain and having at least 95%, 96%, 97%, 98%, 99% or 100% identity to the nucleotide sequence of SEQ ID NO: 48, and/or a nucleotide sequence encoding the light chain and having at least 95%, 96%, 97%, 98%, 99% or 100% identity to the nucleotide sequence of SEQ ID NO: 49;
(d) a nucleotide sequence encoding the heavy chain and having at least 95%, 96%, 97%, 98%, 99% or 100% identity to the nucleotide sequence of SEQ ID NO: 67, and/or a nucleotide sequence encoding the light chain and having at least 95%, 96%, 97%, 98%, 99% or 100% identity to the nucleotide sequence of SEQ ID NO: 68;
(e) a nucleotide sequence encoding the heavy chain and having at least 95%, 96%, 97%, 98%, 99% or 100% identity to the nucleotide sequence of SEQ ID NO: 75, and/or a nucleotide sequence encoding the light chain and having at least 95%, 96%, 97%, 98%, 99% or 100% identity to the nucleotide sequence of SEQ ID NO: 76.

### Uses of the Anti-EGFR Antibodies

The antibody of the present application can be used to treat cancer or tumor, especially solid tumor, in particular glioma, glioblastoma, colorectal cancer, head and neck squamous cell carcinoma, non-small cell lung cancer, gastric cancer, pancreatic cancer, esophageal cancer, breast cancer.

The antibody of the present application can provide enhanced tumor selectivity over normal issue due to their pH-dependent binding. With the cross-reactivity to mouse EGFR, the anti-hEGFR antibody of the present application is suitable for research and development conducted with mice.

### Examples

### Materials & Methods

### Animals.

All mice experiments were conducted following the National Guidelines for Housing and Care of Laboratory Animals in China and were performed under the approved IACUC protocols at the National Institute of Biological Sciences, Beijing. Balb/c and CB-17 SCID were purchased from Charles River.

### Cell Lines.

A-431 cells were provided by Dr. Zhiyuan Zhang (National Institute of Biological Sciences, Beijing); CT26 cells were from the Cell Bank of Type Culture Collection, Chinese Academy of Sciences. The CT26-hEGFR cell line (expressing human EGFR) was generated via transfection followed by G418 selection. A-431 and CT26 cells were cultured with RPMI 1640 medium supplemented with 10% FBS. These cells were cultured at 37° C. in a humidified incubator with a 5% CO₂ atmosphere. The FreeStyle 293F cells were from Life Technologies and were cultured following the manufacturer's instructions.

### Expression and Purification of Proteins.

The ECR of human and mouse EGFR were produced as His₆-Avi-tagged fusion proteins by transient transfection of FreeStyle 293F cells and were purified by affinity chromatography. For full-length IgG antibodies, the coding sequences of the VH and VL were subcloned into a human IgG1 H chain (HC) expression vector and an L chain (LC) expression vector, respectively. 293F cells were co-transfected with the two IgG expression plasmids (HC+LC plasmids) at a 1:1 ratio. After 3-6 days of transfection, the cell culture supernatants were collected for purification of IgG1 via Protein A bead affinity chromatography.

Antibody Library Panning and Screening of pH-Dependent Antibodies.

Then single clones were randomly picked and screened for binding to the corresponding antigen at pH 6.0 and pH 7.4 with ELISA. Similar strategies were used for screening of sub-libraries derived from 14C07 or G5V2, except that pH of acidic buffer was changed from pH 6.0 to pH 6.5 (FIG. S6). Amino acid numbering and CDR definition were conducted based on Kabat, using the abYsis tool (http://www.abysis.org/abysis/).

### ELISA-Based Binding Assays.

For the ELISA-based analysis of IgG antibody binding with the antigens, biotinylated protein antigens (1 μg/ml) were captured with streptavidin (3 μg/ml) (Sigma-Aldrich, Cat #189730) coated 96-well plates (Thermo Fisher Scientific, Cat #449824). Then, serially diluted antibodies were added, and detected by adding an HRP-labeled goat polyclonal anti-Human IgG Fc (Thermo Fisher Scientific, Cat #31413). For the ELISA-based analysis of IgG antibody competition with EGF, biotinylated EGFR protein antigens (1 μg/ml) were captured with streptavidin (3 μg/ml) (Sigma-Aldrich, Cat #189730) coated 96-well plates (Thermo Fisher Scientific, Cat #449824). Then serially diluted antibodies that had been mixed with EGF-mFc (50 nM at pH 6.0-6.5, 15 nM at pH 7.4) were added, followed by detection based on adding an HRP-conjugated anti-mouse Fc secondary antibody (Thermo Fisher Scientific, Cat #31437).

For the ELISA-based analysis of phage display scFv antibody binding with the antigens, biotinylated protein antigens (1 μg/ml) were captured with streptavidin (3 μg/ml) (Sigma-Aldrich, Cat #189730) coated 96-well plates (Thermo Fisher Scientific, Cat #449824). Then serially diluted phage display scFv antibodies were added, and detected by adding an HRP-conjugated anti-M13 antibody (GE Healthcare, Cat #27-9421-01). For the ELISA-based analysis of antibody binding with EGFR and its variants, antibodies (5 μg/ml) were coated on 96-well plates (Thermo Fisher Scientific, Cat #449824). Then serially diluted EGFR and its variants were added, followed by detection based on adding an HRP-labeled His-Tag antibody (Mei5Bio, Cat #MF082-HRP-01). A nonlinear regression model was used to fit ELISA curves; each curve was fitted independently.

Binding kinetic analysis by Surface Plasmon Resonance.

Kinetics analyses of the binding of anti-EGFR antibodies to the ECR of mouse and human EGFR were performed on a Biacore T200 instrument (Biacore, GE Healthcare). Anti-hFc Ab or protein A/G (Thermo Fisher Scientific) was covalently attached to the surface of a CM5 sensor chip using an amine coupling kit (GE Healthcare). Antibodies (1 μg/ml) were captured on the chip and the analytes were then injected at serially diluted concentrations (3200 nM, 1600 nM, 800 nM, 400 nM, 200 nM, 100 nM, 50 nM). Binding kinetics were evaluated using a 1:1 Langmuir binding model. The dissociation constant (KD) values were calculated with a steady-state affinity model using the Biacore T200 evaluation software.

### Flow Cytometry Analysis of Cell Lines.

For examining binding of antibodies with cells, cells were stained with G532, G532^{Ctrl} or Cetuximab followed by staining with a goat polyclonal anti-Human IgG Fc FITC (Thermo Fisher Scientific, Cat #A18830).

### In Vivo Tumor Assays.

6-8-week old mice were inoculated subcutaneously with A-431 or CT26-hEGFR tumor cells (in 100 μL DPBS or medium). CB-17 SCID mice were inoculated with 5×10⁶ A-431 cells, Balb/c mice were inoculated with 1×10⁵ CT26-hEGFR cells. Tumor volume was measured with a digital caliper and calculated using the modified ellipsoid formula ½×(length×width²). Based on similar mean tumor volumes, mice were randomized into groups (n=4-6/group) and received intraperitoneal injection of the examined therapeutic antibodies. When the tumor reached 2 cm in length or when weakness was observed, the mice were sacrificed.

### Immunofluorescence Assays.

For detecting enrichment of antibodies in mice livers and A-431 tumors, CB-17 SCID mice bearing A-431 tumors were sacrificed 48 hr after injection with Control IgG, G532, or G532^{Ctrl}. Then mice livers and tumors were harvested to prepare frozen sections. To detect antibody accumulation, sections were fixed with pre-cold pH 6.5 4% PFA buffer, then sections were stained with FITC-conjugated anti-human IgG secondary antibody or Alexa Fluor 633-conjugated anti-human IgG secondary antibody (Thermo Fisher Scientific, Cat #A-21091). To evaluate tumor vasculature, sections were fixed with pre-cold pH 6.5 4% PFA buffer, then sections were incubated with Rat anti-mouse CD31 (BD Pharmingen, Cat #557355) prior to staining with Alexa Fluor 647-conjugated anti-Rat IgG secondary antibody (Thermo Fisher Scientific, Cat #A-21247). Antibody enrichment was assessed based on the fluorescence intensity for FITC (Thermo Fisher Scientific, Cat #A18830). Immunofluorescence intensities were analyzed with ImageJ.

### Statistics.

GraphPad Prism 6 was used for comparisons throughout the manuscript, with p values indicated in the relevant Figures. Two-way ANOVA or two-tailed unpaired Student's t-tests were applied. Two-way ANOVA was applied for analyzing antitumor activity. p values<0.05 were regarded as statistically significant.

### Example 1. Selection of a pH-Dependent Anti-EGFR Antibody with Ligand-Binding Activity from a Non-Immune Human Antibody Library

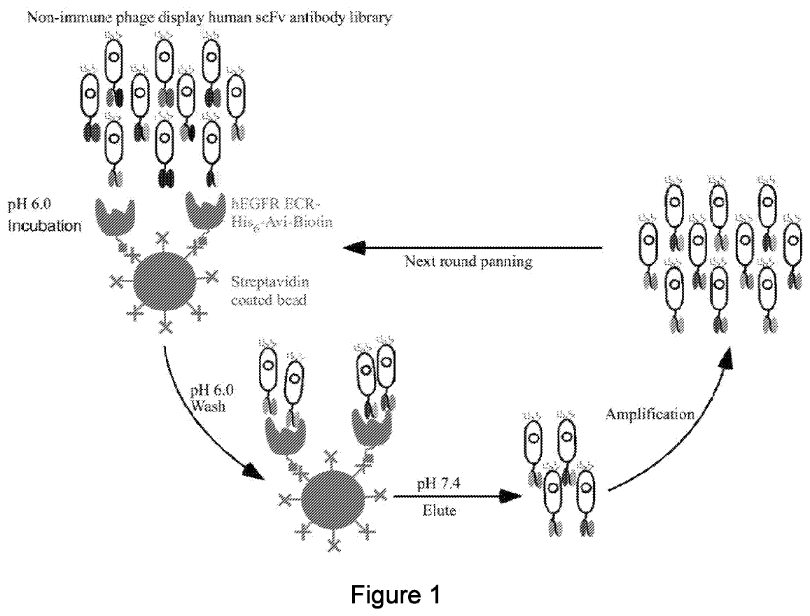

To identify pH-dependent human antibodies against hEGFR (SEQ ID NO: 1), a recombinant protein hEGFR ECR-His6-Avi-Biotin was produced by fusing the extracellular domain (ECR) of hEGFR (residues Leu25-Lys642; SEQ ID NO: 2) with a His₆-Avi tag and biotinylated using BirA ligase. The hEGFR ECR fusion protein was used as an antigen to select pH-dependent antibodies from a non-immune phage display human scFv antibody library (library size: 1.1×10¹⁰) (Li, D. et al., (2017). A potent human neutralizing antibody Fe-dependently reduces established HBV infections. Elife 6, 26738). To screen for pH-dependent antibodies, the non-immune phage display library was incubated with the hEGFR ECR fusion protein in a pH 6.0 solution, washed with pH 6.0 washing buffer, followed by elution with pH 7.4 buffer (FIG. 1). Single clones were then randomly picked and screened for binding to the hEGFR ECR at pH 6.0 and pH 7.4 using ELISA.

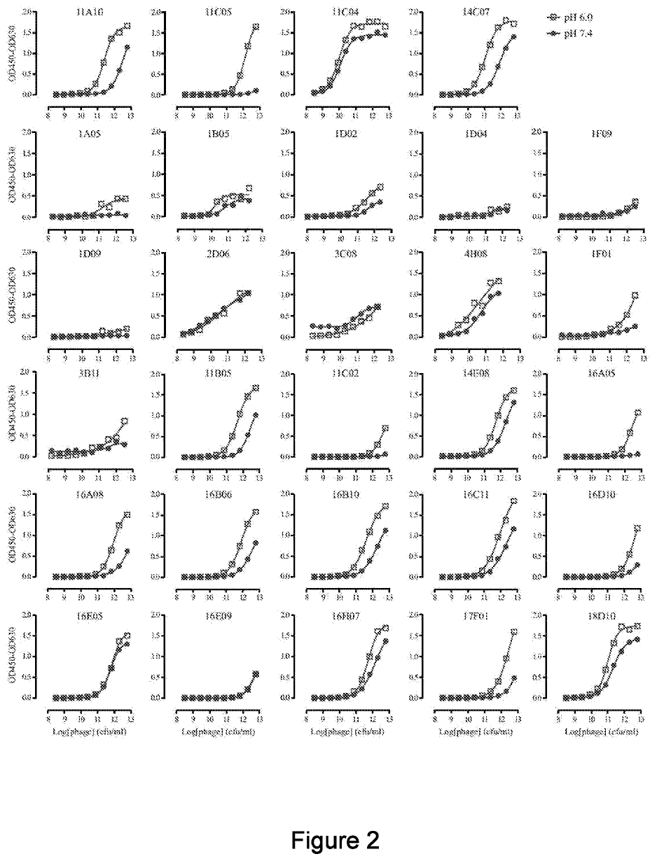

A total of 31 phage display scFv antibodies with unique sequences were thusly selected, and both their binding affinity and pH-dependency were evaluated (FIG. 2). Sequences of representative antibodies, including 14C07, 11A10, 11C04 and 11C05 are shown in Table 1 below. Based on the results as shown in FIG. 2, 14C07 and 11A10 were selected for their relatively high binding affinity and pH-dependency, and both were converted into the full-length human IgG1 format.

TABLE 1
Anti-EGFR antibodies selected from phage display library

| | Variable region amino acid | |
|---|---|---|
| Description | sequence (SED ID NO) | CDRs (SEQ ID NOs) |
| 14C07 VH | QVQLVQSGAEVKRPGSSVKVSCKASGGTFS | HCDR1: SYAIS (SEQ ID NO: 11) |
| | SYAISWVRQAPGQGLEWMGGIIPIFGTANY | HCDR2: GIIPIFGTANYAQKFQG (SEQ |
| | AQKFQGRVTITADESTSTAYMELSSLRSEDT | ID NO: 12) |
| | AVYYCARDSSSGWYDYFDYWGQGTLVTV | HCDR3: DSSSGWYDYFDY (SEQ ID |
| | SS (SEQ ID NO: 5) | NO: 13) |
| 14C07 VL | QPVLTQPPSVSAAPGAMARITCESPDIGSKS | LCDR1: ESPDIGSKSVQ (SEQ ID NO: |
| | VQWYQQRPGQAPILVIYYDDDRPSGIPERFS | 14) |
| | GSNSGNTAALTINWVEPEDEADYYCQAWD | LCDR2: YDDDRPS (SEQ ID NO: 15) |
| | DSDPVVFGGGTQLTVL (SEQ ID NO: 6) | LCDR3: QAWDDSDPVV (SEQ ID NO: |
| | | 16) |
| 11A10 VH | EVQLVQSGAEVKKPGESLMISCKGSGYSFT | HCDR1: NHWIG (SEQ ID NO: 23) |
| | NHWIGWVRQMPGKGPEWMGIINPRDSDA | HCDR2: IINPRDSDARYSPSFRG (SEQ |
| | RYSPSFRGQVTFSVDRSTNTAYLQWTSLKP | ID NO: 24) |
| | SDTGMYFCARRDIVVVPAATPSYYYYGMD | HCDR3: |
| | VWGQGTTVTVSS (SEQ ID NO: 17) | RDIVVVPAATPSYYYYGMDV (SEQ ID |
| | | NO: 25) |
| 11A10 VL | DIQLTQSPSSLSASVGDSVTITCRPROSSSRF | LCDR1: RPRQSSSRFVN (SEQ ID NO: |
| | VNWYQHKPGKAPKLLISNASDLQHGVPSR | 26) |
| | FSGRGAGTEFTLTIDSLQPDDVATYYCEQSY | LCDR2: NASDLQH (SEQ ID NO: 27) |
| | RFPHMFGQGTKVEIK (SEQ ID NO: 18) | LCDR3: EQSYRFPHM (SEQ ID NO: 28) |
| 11C04 VH | EVQLVQSGAEVKKPGASVKVSCKASGYTFT | HCDR1: SYYMH (SEQ ID NO: 31) |
| | SYYMHWVRQAPGQGLEWMGIINPSGGSTS | HCDR2: IINPSGGSTSYAQKFQG (SEQ |
| | YAQKFQGRVTMTRDTSTSTVYMELSSLRSE | ID NO: 32) |
| | DTAVYYCARDLEMAYYDILYGMDVWGQG | HCDR3: DLEMAYYDILYGMDV (SEQ |
| | TLVTVSS (SEQ ID NO: 29) | ID NO: 33) |
| 11C04 VL | QSALTQPASVSGSAGQSITISCTGTTSDIGRY | LCDR1: TGTTSDIGRYDYVS (SEQ ID |
| | DYVSWYQQHPGQVPKLMIYDVSNRPSGVS | NO: 34) |
| | DRFSGSKSGNTASLTISGLQTEDEADYYCSS | LCDR2: DVSNRPS (SEQ ID NO: 35) |
| | YTTSGTRVFGGGTKLTVL (SEQ ID NO: 30) | LCDR3: SSYTTSGTRV (SEQ ID NO: 36) |
| 11C05 VH | EVQLVQSGAEVKKPGSSVKVSCKASGGTFS | HCDR1: SYAIS (SEQ ID NO: 11) |
| | SYAISWVRQAPGQGLEWMGGIIPIFGTANY | HCDR2: GIIPIFGTANYAQKFRG (SEQ |
| | AQKFRGRVTITADESTSTAYMELSSLRSEDT | ID NO: 39) |
| | AVYYCARDRDVWFGELLSYYYYGMDVW | HCDR3: DRDVWFGELLSYYYYGMDV |
| | GQGTTVTVSS (SEQ ID NO: 37) | (SEQ ID NO: 40) |
| 11C05 VL | QSVVTQPPSVSAAPGQKVTISCSGSSSNIGN | LCDR1: SGSSSNIGNSYVS (SEQ ID |
| | SYVSWYQQLPGTAPKLLIYDNNKRPSGIPD | NO: 41) |
| | RFSGSKSGTSATLGITGLQTGDEADYYCAT | LCDR2: DNNKRPS (SEQ ID NO: 42) |
| | WDSSLSAVVFGGGTKLTVL (SEQ ID NO: 38) | LCDR3: ATWDSSLSAVV (SEQ ID NO: |
| | | 43) |

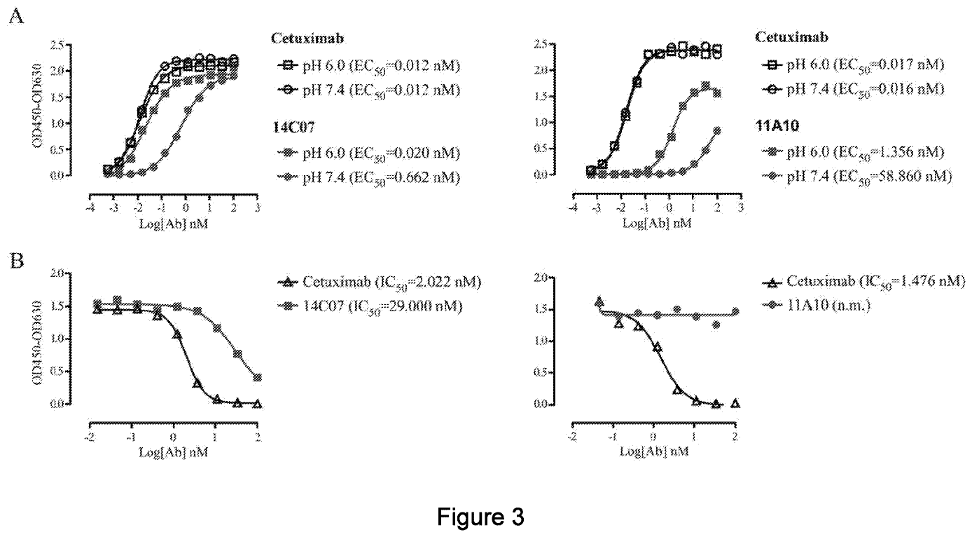

Next, the binding affinity of 14C07, 11A10 were compared with the FDA-approved therapeutic anti-EGFR mAb Cetuximab for hEGFR at pH 6.0 and pH 7.4. The antibodies in human IgG1 form at the indicated concentrations were added to 96-well plates coated with hEGFR ECR, followed by extensive washing. Then antibody-hEGFR binding was detected with an HRP-conjugated anti-hFc secondary antibody. The EC₅₀ values are shown in FIG. 3A. It was found that both 14C07 and 11A10 exhibited potent pH-dependent binding, with dramatically higher binding affinity at pH 6.0 than at pH 7.4 (FIG. 3A). In contrast, the binding affinity of Cetuximab was quite similar at the two pH values (FIG. 3A).

The ligand-blocking ability of 14C07, 11A10, and Cetuximab was also evaluated in assays with EGFR and one of its ligands (EGF) at pH 6.0 with ELISA. EGF-mFc was mixed with the antibodies in human IgG1 form at the indicated concentrations, and added to 96-well plates coated with hEGFR ECR, followed by extensive washing. Then EGF-hEGFR binding was measured using an HRP-conjugated anti-mFc secondary antibody. Both 14C07 and Cetuximab could block EGFR-EGF binding at pH 6.0 in a dose-dependent manner (FIG. 31B). In contrast, 11A10 did not block EGFR-EGF binding (FIG. 31B).

Given that binding affinity of 14C07 and Cetuximab is comparable at pH 6.0 (assessed with ELISA), the dramatic difference detected for Cetuximab and 14C07 in blocking EGF-EGFR binding strongly suggests that Cetuximab and 14C07 did not have identical binding epitopes (FIGS. 3A and 31B). Considering that inhibition of ligand binding to therapeutically block an oncogenic signal is one of the known MOAs with the anti-EGFR antibodies used in clinical treatment, 14C07 was selected for further investigation.

### Example 2. Binding of 14C07 to EGFR Variants with H370A and H1433A

A previously reported crystal structure of EGFR/EGF complex revealed that two histidines (H370 and H433) in EGFR's domain III (residues Lys335-Lys538) participate in the EGFR-EGF interaction (Ogiso, H. et al., 2002). This motivated our speculation that EGFR H370 and H433 may participate in EGFR-14C07 binding.

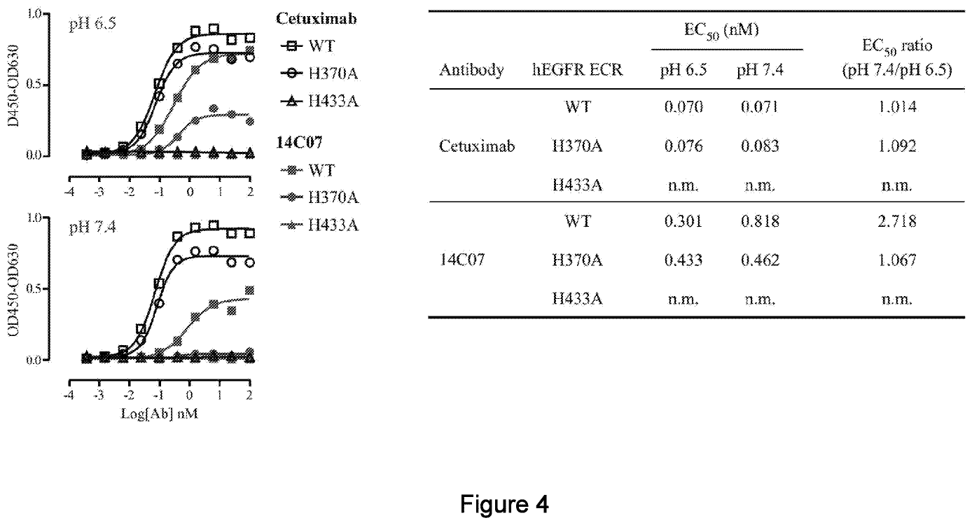

In this example, two hEGFR ECR variants containing H370A and H433A mutations, respectively were generated. The binding of both Cetuximab and 14C07 with wild type (WT) hEGFR ECR and the mutant variants at both pH 6.5 and pH 7.4 were assayed in a similar manner as described in Example 1. The results are shown in FIG. 4, with the EC₅₀ values and binding pH-dependency (EC₅₀ ratio (pH 7.4/pH 6.5)) shown in the table of FIG. 4.

As shown in FIG. 4, 14C07 binding with EGFR was greatly impaired by both EGFR mutants with either H370A or H433A mutation, whereas Cetuximab binding with EGFR was impaired by the H433A mutation but not the H370A mutation (FIG. 4). The pH-dependency of binding between 14C07 and EGFR ECR H370A was more than 2-fold weaker than the pH-dependency between 14C07 and WT EGFR ECR (FIG. 4).

These findings provide four insights into 14C07's epitope on the hEGFR ECR. 1) The epitopes of 14C07 and Cetuximab are not identical. 2) 14C07 can block EGF-EGFR binding, and residues H370 and H433 in EGFR's domain III participate in EGFR-14C07 binding, indicating that the binding epitope of 14C07 is located on EGFR domain III. 3) EGFR H370 affects both the binding affinity and the pH-dependency of 14C07-EGFR binding. 4) H433A abolishes 14C07-EGFR binding, indicating that H433 may exert a stronger influence on 14C07-EGFR pH-dependent binding than H370.

### Example 3. Cross-Reactivity of 14C07 with Human and Mouse EGFR

Considering H370 and H433 residues are conserved between hEGFR and mEGFR, the cross-reactive binding of 14C07 to hEGFR (SEQ ID NO: 1) and mEGFR (SEQ ID NO: 3) was tested.

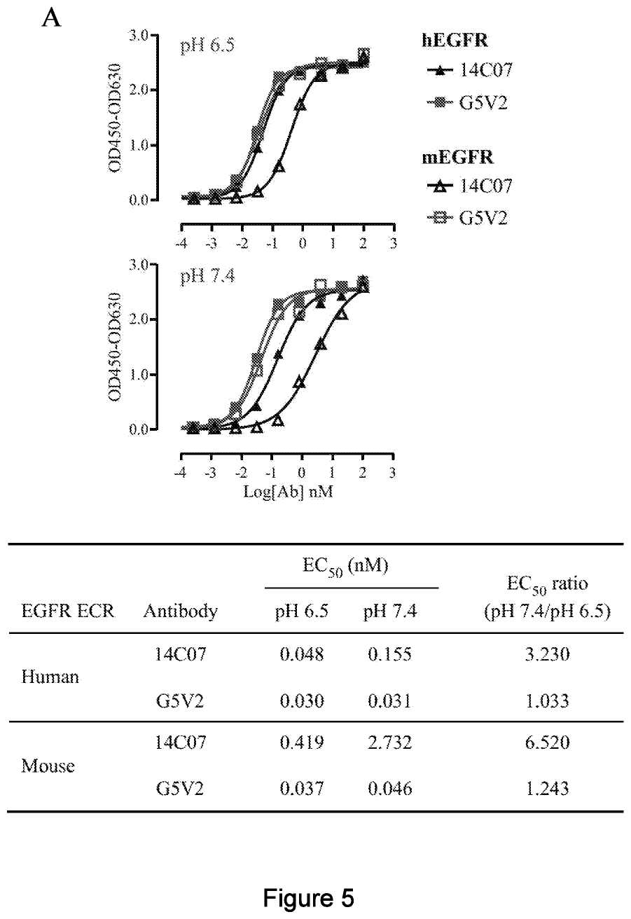

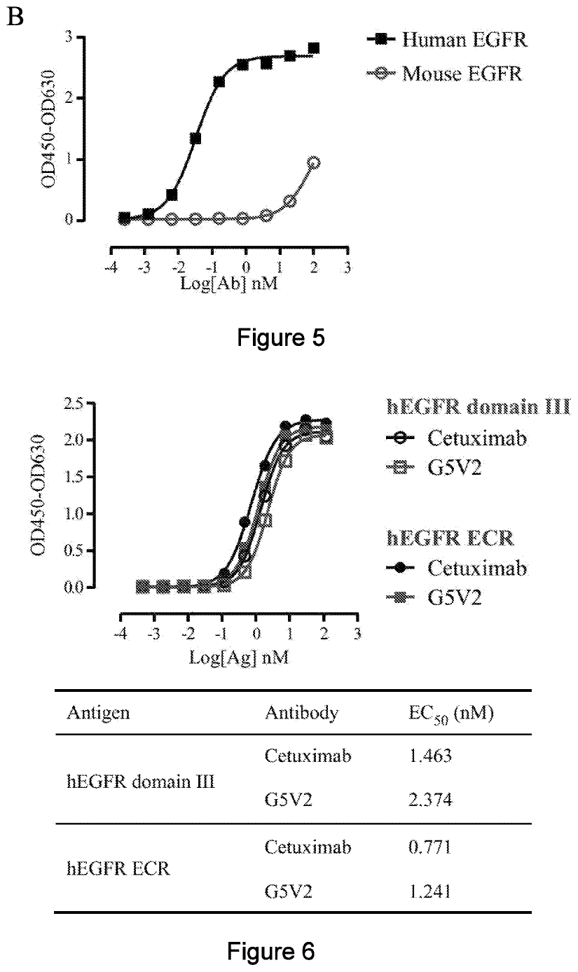

14C07 in human IgG1 form at the indicated concentrations (100, 20, 4, 0.8, 0.16, 0.032, 0.0064, 0.00128, 0.00026, 0.00005, 0.00001 nM) were added to 96-well plates coated with hEGFR ECR (SEQ ID NO: 2) or mEGFR ECR (SEQ ID NO: 4). Then antibody binding with hEGFR or mEGFR was measured with an HRP-conjugated anti-hFc secondary antibody. The EC₅₀ values and binding pH-dependency (EC₅₀ ratio (pH 7.4/pH 6.5)) are shown in the table of FIG. 5A. Assessing 14C07 binding with hEGFR and mEGFR showed that 14C07 bound with both hEGFR and mEGFR, consistently doing so in a dose-dependent and pH-dependent manner (FIG. 5A).

Similar assay was also conducted with Cetuximab. Distinct from 14C07, Cetuximab elicited only weak cross-reactive binding with mEGFR (FIG. 5B).

### Example 4. Generation of an Anti-EGFR Antibody G5V2 with Cross-Reactivity to Both Human and Mouse EGFR by Antibody Affinity Maturation of 14C07

To generate pH-dependent anti-EGFR antibodies with higher binding affinity than 14C07, point mutation scanning was used to select permissive amino acid sites in 6 CDRs of 14C07. Sub-libraries of 14C07 containing random mutations of these selected permissive amino acid sites were constructed. To screen the 14C07 sub-libraries, binding affinity of the library antibodies were tested at pH 6.5 and compared with 14C07.

An anti-EGFR antibody G5V2 with improved cross-reactivity was obtained. G5V2 showed improved binding to both human and mouse EGFR over 14C07. In a binding affinity test performed in a similar manner as Example 3, G5V2's binding affinity for hEGFR and mEGFR at pH 6.5 (EC₅₀ ratio, mEGFR/hEGFR=1.233) were much closer than 14C07's binding affinity for hEGFR and mEGFR at pH 6.5 (EC₅₀ ratio, mEGFR/hEGFR=8.729) (FIG. 5A). However, the difference in affinity between the pH 7.4 and pH 6.5 conditions was less pronounced for G5V2 as compared to 14C07 (FIG. 5A).

As compared to Cetuximab, G5V2 exhibited comparable binding affinity with EGFR domain III and the EGFR ECD (FIG. 6).

Sequences of variable regions of G5V2 are shown in Table 2 below.

TABLE 2
Sequences of G5V2 antibody

| | Variable region amino acid | |
|---|---|---|
| Description | sequence (SED ID NO) | CDRs (SEQ ID NOs) |
| G5V2 VH | QVQLVQSGAEVKRPGSSVKVSCKASGGISD | HCDR1: GSAIS (SEQ ID NO: 50) |
| | GSAISWVRQAPGQGLEWMGGIYFFNGNTN | HCDR2: GIYFFNGNTNYAQKFQG |
| | YAQKFQGRVTITADESTSTAYMELSSLRSED | (SEQ ID NO: 51) |
| | TAVYYCARDSSLGYYDYFDYWGQGTLVTV | HCDR3: DSSLGYYDYFDY (SEQ ID |
| | SS (SEQ ID NO: 44) | NO: 52) |
| G5V2 VL | QPVLTQPPSVSAAPGQTARITCGGNNIGEK | LCDR1: GGNNIGEKYVQ (SEQ ID NO: |
| | YVQWYQQRPGQAPVLVIYYDDDRPSGIPER | 53) |
| | FSGSNSGNTAALTINSVEAGDEADYYCQAW | LCDR2: YDDDRPS (SEQ ID NO: 15) |
| | DDYDPVVFGGGTQLTVL (SEQ ID NO: 45) | LCDR3: QAWDDYDPVV (SEQ ID NO: |
| | | 54) |

### Example 5. Identification of a Hotspot Increasing the pH-Dependency of G5V2-hEGFR Binding by a G5V2-hEGFR Structure Model

In this example, candidate hotspots affecting pH-dependent binding between G5V2 and EGFR was investigated by structural analysis.

### Construction of a G5V2-hEGFR Complex Structure Model.

A G5V2 scFv structure model was generated by using Abodybuilder (Leem, J. et al., (2016). ABodyBuilder: Automated antibody structure prediction with data-driven accuracy estimation. MAbs 8, 1259-1268).

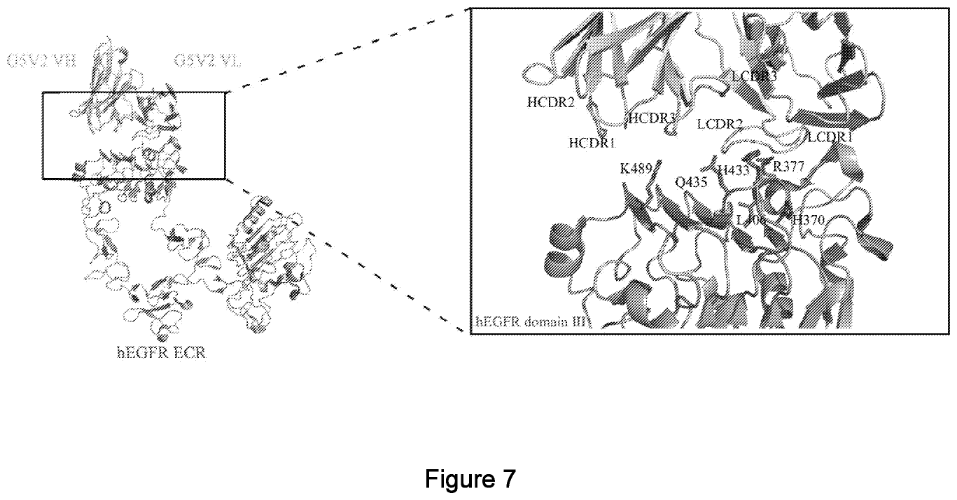

Then a complex structure model of G5V2-hEGFR (FIG. 7) was manually docked using PyMOL with the help of a previously reported crystal structure of a Cetuximab/EGFR complex (PDB ID: 1YY9) (Ogiso, H. et al., 2002; Li, S. et al., 2005). Specifically, the Cetuximab/EGFR complex structure (PDB ID: 1YY9) and the G5V2 (in scFv form) structure model as generated were opened using Abodybuider in PyMOL. After hiding Cetuximab in the Cetuximab/EGFR complex, the position and orientation of G5V2 and hEGFR were adjusted to form a model of G5V2-hEGFR complex using the 3-Button Editing mode, based on the following steps:

1) G5V2 exhibited comparable binding affinity with the hEGFR ECR and hEGFR domain III (FIG. 6), indicating that the binding epitope of G5V2 is likely located on hEGFR domain III. Thus, G5V2 was moved to face hEFGR domain III.

2) The EGFR H433A mutation abolished 14C07-hEGFR binding, while the EGFR H370A mutation reduced 14C07-hEGFR binding (FIG. 1C), indicating that the H433 and H370 residues are likely located on the binding interface between hEGFR and 14C07 (the parent antibody of G5V2). Thus, EGFR H370 and H433 were allowed to present at the binding interface, ensuring that EGFR H433 is positioned closer to G5V2 than EGFR H370 (since EGFR H433 contributes more to the binding than EGFR H370 according to EGFR H433A mutation exhibiting stronger impact on decreasing binding affinity than EGFR H370A mutation (FIG. 4)).

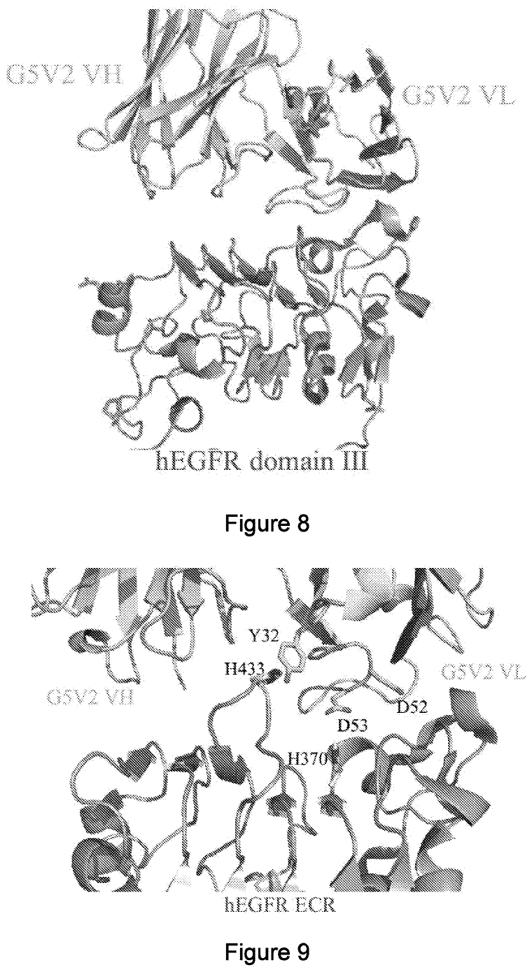

3) G5V2 exhibits comparable binding affinity to human and mouse EGFR (FIG. 5A), indicating that the binding epitope of G5V2 conserves the same or highly similar residues between human and mouse EGFR. Thus, the position of G5V2 and hEGFR was adjusted to avoid the presentation of non-conserved residues between human EGFR and murine EGFR to the greatest extent (FIG. 8).

Also concerned was that 14C07 can block EGFR-EGF binding, indicating that its variant G5V2 interacts with EGFR at the EGFR-EGF binding interface.

As shown in FIG. 8, five amino acids, namely R377, L406, H433, Q435, and K489 were identified as residues located at the binding surface of G5V2-hEGFR complex structure model.

### Confirmation of Epitope Residues by Alanine Substitution

To confirm the role of the five residues potentially involved in epitope binding as indicated by the G5V2-hEGFR complex structure model, variants of hEGFR were generated by analine substitution and binding of G5V2 with said variants was determined by ELISA.

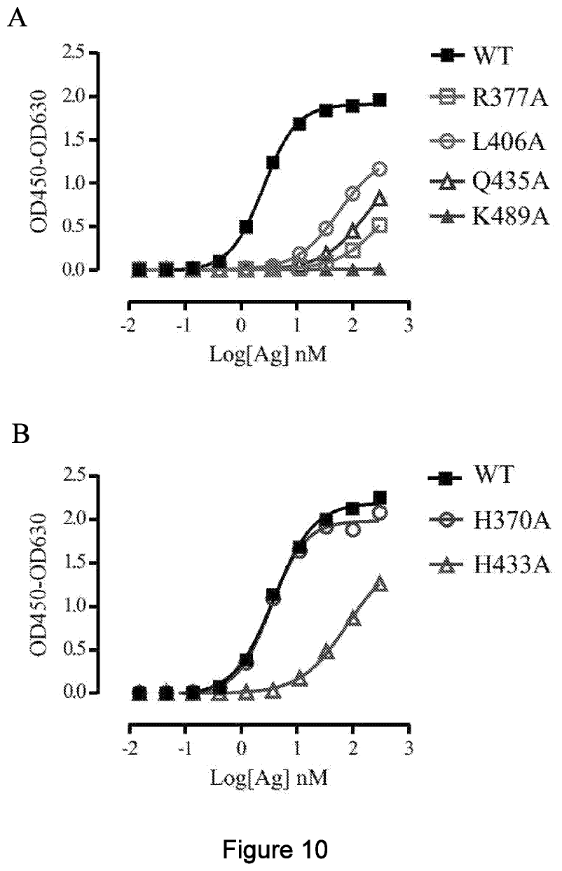

The hEGFR ECR and its variants (with H370A, R377A, L406A, H433A, Q435A, or K489A, respectively) at the indicated concentrations as shown in FIG. 10A-B were added to 96-well plates coated with G5V2 (in human IgG1 form), followed by extensive washing. Then, antibody-hEGFR binding was detected with an HRP-labeled His-Tag antibody secondary antibody.

Supporting the reliability of the conformational epitope indicated by the G5V2-EGFR structure model, ELISA-based binding analysis showed that alanine substitution of 5 amino acids, which are located at the binding interface revealed by the G5V2-hEGFR complex structure model (R377A, L406A, H433A, Q435A, and K489A), significantly reduced the binding of G5V2 to hEGFR (FIGS. 7, 9A and 9B).

ELISA assessment of G5V2 binding with WT hEGFR ECR and the mutant variants containing H433A or H370A mutations showed that G5V2-hEGFR binding was impaired by the H433A mutation but not the H370A mutation (FIG. 10B). Consistent with this result, examining the G5V2-hEGFR the complex structure model indicated close contact between G5V2 LCDR1 Y32 and hEGFR H433, yet relatively weak contact between G5V2 LCDR2 D52D53 and hEGFR H370 (FIG. 9).

Identification of a Hotspot on G5V2 Determining pH-Dependent Binding to hEGFR

Previous studies have shown that protonation of ionizable residues like histidine can contribute to pH-dependent binding. pH-dependent binding between G5V2 and EGFR may be increased by creating interaction of histidine (H) with acidic amino acids such as aspartate (D) or glutamate (E)).

H433 was targeted for engineering of pH-dependency, as H433 was identified as a residue involved in G5V2-hEGFR binding by the complex structure model and G5V2-hEGFR binding was impaired by the H433A mutation. New H-D/E interaction was introduced between hEGFR H433 and G5V2 LCDR1 Y32 with a purpose of enhancing the pH-dependency of G5V2.

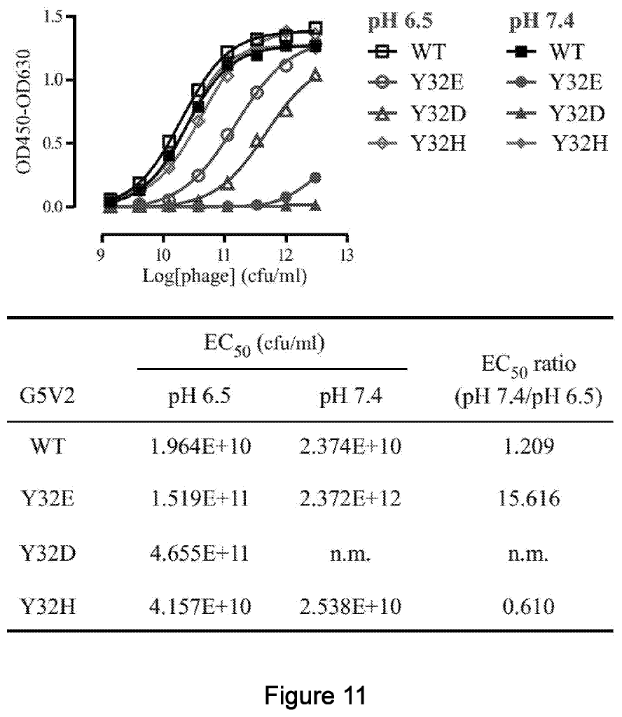

Three G5V2 variants containing LCDR1 Y32E, Y32D, or Y32H mutations were generated in phage display Fab antibody form, and the binding of these antibodies was compared with hEGFR ECR at pH 6.5 and pH 7.4. The phage display Fab antibodies at the indicated concentrations as shown in FIG. 11 were added to coated hEGFR ECR, followed by extensive washing. Then antibody-hEGFR binding was analyzed with an HRP-conjugated anti-M13 secondary antibody.

As shown in FIG. 11, the Y32H mutation did not significantly affect binding pH-dependency or affinity. In sharp contrast, the Y32E and Y32D mutations dramatically increased binding pH-dependency (FIG. 11). This result indicates that the close interaction of G5V2 LCDR1 Y32-EGFR H433 suggested by the G5V2-EGFR structure model is a hotspot determining G5V2-EGFR pH-dependent binding, and by introducing a H-D/E interaction between G5V2 LCDR1 Y32 and EGFR H433, the pH-dependency of G5V2-EGFR binding was greatly increased.

### Example 6. Generation of a pH-Dependent Anti-EGFR Antibody (G532) Based on Structure-Guided Engineering of G5V2

G5V2 Y32E was selected for further engineering in this example, owing to its ~13-fold higher pH-dependent binding over WT G5V2 and its ~3-fold higher binding affinity over G5V2 Y32D (FIG. 11). Note that the Y32E mutation decreased the binding affinity of G5V2 at pH 6.5 by ~8-fold (FIG. 11), suggesting that the antitumor activity of G5V2 Y32E is likely weaker than G5V2, owing to its lower binding affinity than G5V2. Given that the goal of invention is generating a pH-dependent anti-EGFR antibody with improved binding affinity while maintaining adequate pH-dependency to support selective binding in the acidic tumor microenvironment, the inventors next focused on HCDR2, which was shown by structural analysis as having no obvious impact on pH-dependency (FIG. 7).

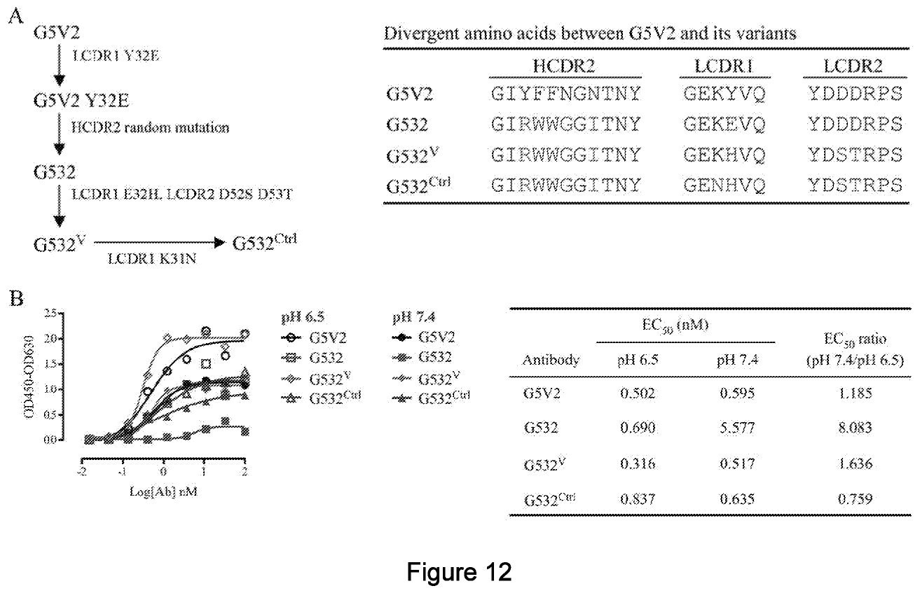

The inventors constructed and screened a sub-library of G5V2 Y32E with random HCDR2 mutations. A candidate pH-dependent anti-EGFR antibody (G532) was selected from the sub-library based on its desirable binding affinity at pH 6.5 and its obvious pH-dependent binding with hEGFR ECR (EC₅₀ ratio, pH 7.4/pH 6.5=8.083) (FIGS. 12A and 12B). The binding assay was performed similarly as in Example 1.

To assess the specific impact of H-D/E interactions on the observed pH-dependent binding, a G532 variant G532^{V} was generated by replacing E32 in LCDR1 of G532 with histidine (H), and replacing D52 and D53 in LCDR2 of G532 with serine (S) and threonine (T), respectively, with a purpose of disrupting two H-D/E interactions (G532 LCDR1 E32-EGFR H433, G532 LCDR2 D52D53-EGFR H370) between G532 and EGFR as indicated by the G5V2-hEGFR complex structure model. As shown in FIGS. 12A and 12B, G532^{V} had ~5-fold lower binding pH-dependency with hEGFR ECR than G532. Notably, G532^{V} retained weak binding pH-dependency (EC₅₀ ratio, pH 7.4/pH 6.5=1.636) (FIG. 12B).

Further, amino acids positioned close to G532^{V} LCDR1 H32 were mutated into amino acids with smaller sidechains, aiming to generate a G532 variant with abolished binding selectivity at pH6.5. Specifically, a non-pH-dependent G532 variant, G532^{Ctrl} was generated by introducing LCDR1 K31N mutation into G532^{V}. As shown in FIG. 12A-B, G532^{Ctrl} has no binding selectivity at pH6.5 (EC₅₀ ratio, pH 7.4/pH 6.5=0.759).

Sequences of G532, G532^{V} and G532^{Ctrl} are shown in Table 3 below.

TABLE 3
Sequences of antibodies G532, G532^{V} and G532^{Ctrl}

| | Variable region amino acid | |
|---|---|---|
| Description | sequence (SED ID NO) | CDRs (SEQ ID NOs) |
| G532 VH | QVQLVQSGAEVKRPGSSVKVSCKASGGISD | HCDR1: GSAIS (SEQ ID NO: 50) |
| | GSAISWVRQAPGQGLEWMGGIRWWGGIT | HCDR2: GIRWWGGITNYAQKFQG |
| | NYAQKFQGRVTITADESTSTAYMELSSLRSE | (SEQ ID NO: 61) |
| | DTAVYYCARDSSLGYYDYFDYWGQGTLVT | HCDR3: DSSLGYYDYFDY (SEQ ID |
| | VSS (SEQ ID NO: 55) | NO: 52) |
| G532 VL | QPVLTQPPSVSAAPGQTARITCGGNNIGEK | LCDR1: GGNNIGEKEVQ (SEQ ID NO: |
| | EVQWYQQRPGQAPVLVIYYDDDRPSGIPER | 62) |
| | FSGSNSGNTAALTINSVEAGDEADYYCQAW | LCDR2: YDDDRPS (SEQ ID NO: 15) |
| | DDYDPVVFGGGTQLTVL (SEQ ID NO: 56) | LCDR3: QAWDDYDPVV (SEQ ID NO: |
| | | 54) |
| G532Y VH | QVQLVQSGAEVKRPGSSVKVSCKASGGISD | HCDR1: GSAIS (SEQ ID NO: 50) |
| | GSAISWVRQAPGQGLEWMGGIRWWGGIT | HCDR2: GIRWWGGITNYAQKFQG |
| | NYAQKFQGRVTITADESTSTAYMELSSLRSE | (SEQ ID NO: 61) |
| | DTAVYYCARDSSLGYYDYFDYWGQGTLVT | HCDR3: DSSLGYYDYFDY (SEQ ID |
| | VSS (SEQ ID NO: 63) | NO: 52) |
| G532V VL | QPVLTQPPSVSAAPGQTARITCGGNNIGEK | LCDR1: GGNNIGEKHVQ (SEQ ID NO: |
| | HVQWYQQRPGQAPVLVIYYDSTRPSGIPER | 69) |
| | FSGSNSGNTAALTINSVEAGDEADYYCQAW | LCDR2: YDSTRPS (SEQ ID NO: 70) |
| | DDYDPVVFGGGTQLTVL (SEQ ID NO: 64) | LCDR3: QAWDDYDPVV (SEQ ID NO: |
| | | 54) |
| G532Ctrl VH | QVQLVQSGAEVKRPGSSVKVSCKASGGISD | HCDR1: GSAIS (SEQ ID NO: 50) |
| | GSAISWVRQAPGQGLEWMGGIRWWGGIT | HCDR2: GIRWWGGITNYAQKFQG |
| | NYAQKFQGRVTITADESTSTAYMELSSLRSE | (SEQ ID NO: 61) |
| | DTAVYYCARDSSLGYYDYFDYWGQGTLVT | HCDR3: DSSLGYYDYFDY (SEQ ID |
| | VSS (SEQ ID NO: 71) | NO: 52) |
| G532 Ctrl VL | QPVLTQPPSVSAAPGQTARITCGGNNIGEN | LCDR1: GGNNIGENHVQ (SEQ ID NO: |
| | HVQWYQQRPGQAPVLVIYYDSTRPSGIPER | 77) |
| | FSGSNSGNTAALTINSVEAGDEADYYCQAW | LCDR2: YDSTRPS (SEQ ID NO: 70) |
| | DDYDPVVFGGGTQLTVL (SEQ ID NO: 72) | LCDR3: QAWDDYDPVV (SEQ ID NO: |
| | | 54) |

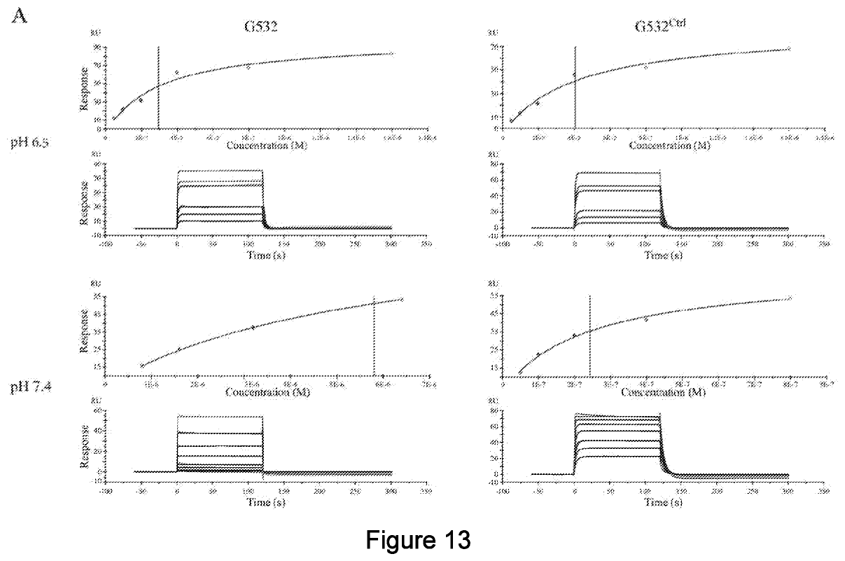

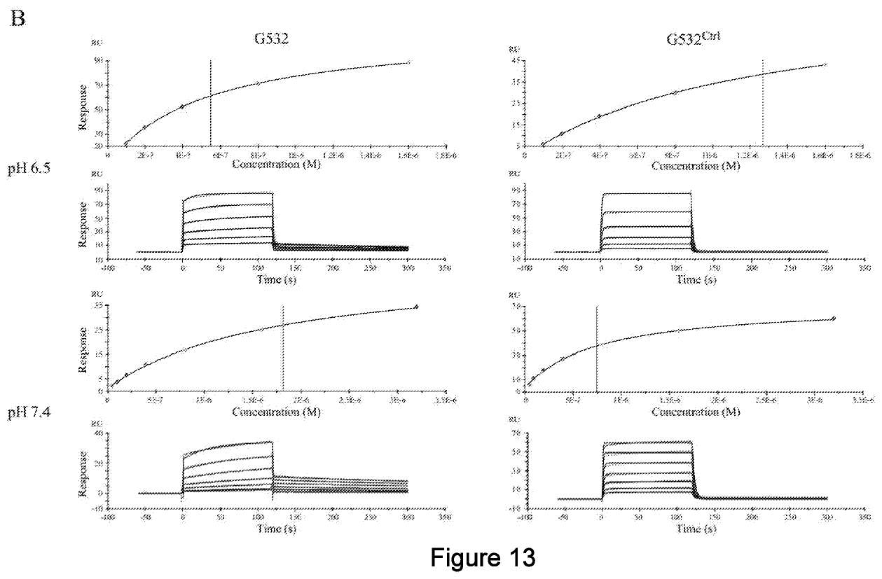

Next, SPR was used to assess binding affinity of G532, and G532^{Ctrl} binding with hEGFR and with mEGFR. G532 exhibited potent binding selectivity for both hEGFR and mEGFR at pH 6.5 than pH 7.4; in contrast, G532^{Ctrl} exhibited similar binding affinity for both hEGFR and mEGFR at pH 6.5 and pH 7.4 (Tablet and FIG. 13). In Table 4, the dissociation constant (KD) values were calculated with a steady-state affinity model using Biacore T200 evaluation software.

TABLE 4
SPR analysis of the binding affinity and pH-dependency of G532 and G532^{Ctrl}.

| | Human EGFR | | | Mouse EGFR | | |
|---|---|---|---|---|---|---|
| | KD (M) | KD (M) | KD ratio | KD (M) | KD (M) | KD ratio |
| | pH 6.5 | pH 7.4 | (pH 7.4/6.5) | pH 6.5 | pH 7.4 | (pH 7.4/6.5) |
| G532 | 2.94E−07 | 3.90E−06 | 13.262 | 5.47E−07 | 1.81E−06 | 3.309 |
| G532^{Ctrl} | 4.06E−07 | 2.43E−07 | 0.599 | 1.27E−06 | 7.43E−07 | 0.586 |

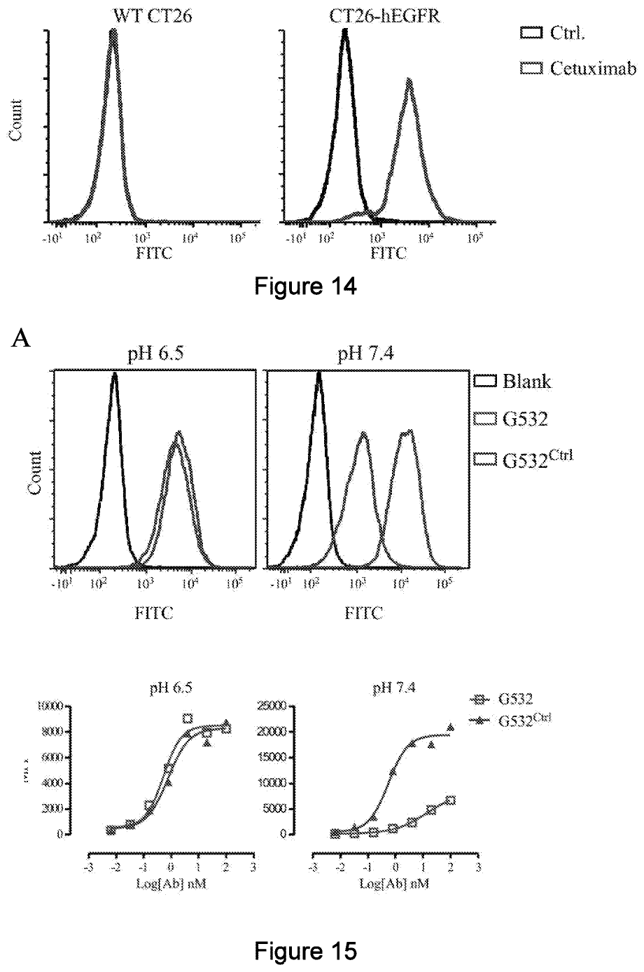

The binding affinity and selectivity of G532, and G532^{Ctrl} were also compared in FACS with cell lines (A-431 cell line and a mouse colon carcinoma cell line expressing hEGFR (CT26-hEGFR)) expressing full-length EGFR. The expression of EGFR in the cell lines were determined by incubating with Cetuximab (200 nM) prior to staining with FITC-conjugated anti-human IgG secondary antibody. Then human EGFR expression was determined based on the fluorescence intensity for FITC and shown in FIG. 14.

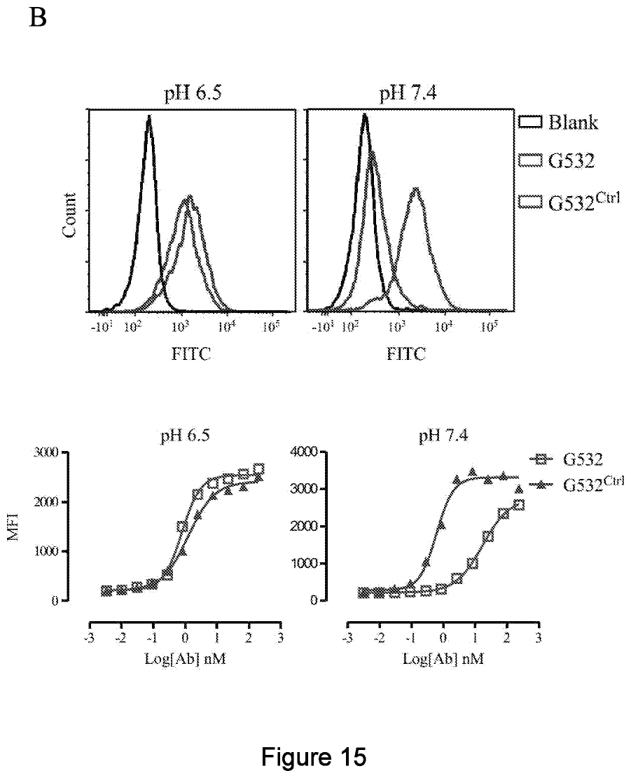

For the test of G532, and G532^{Ctrl}, the cells were incubated with the antibodies prior to staining with FITC-conjugated anti-human IgG secondary antibody. Antibody binding to cells was then assessed based on the fluorescence intensity for FITC. G532 exhibited obvious pH-dependent binding with both A-431 and CT26-hEGFR cells; in contrast, G532^{Ctrl} exhibited similar binding affinity for both tested cell lines at pH 6.5 and pH 7.4 (FIG. 15A-B, Table 5).

TABLE 5
EC₅₀ values of G532 and G532Ctrl for binding with A-431 cells or
CT26-hEFGR cells based on the FACS analysis shown in FIG.3C-D

| | A-431 | | | CT26-hEGFR | | |
|---|---|---|---|---|---|---|
| | EC₅₀ (nM) | EC₅₀ (nM) | EC₅₀ ratio | EC₅₀ (nM) | EC₅₀ (nM) | EC₅₀ ratio |
| | pH 6.5 | pH 7.4 | (pH 7.4/6.5) | pH 6.5 | pH 7.4 | (pH 7.4/6.5) |
| G532 | 0.528 | 13.740 | 26.042 | 0.787 | 15.240 | 19.357 |
| G532^{Ctrl} | 0.771 | 0.554 | 0.718 | 1.253 | 0.587 | 0.469 |

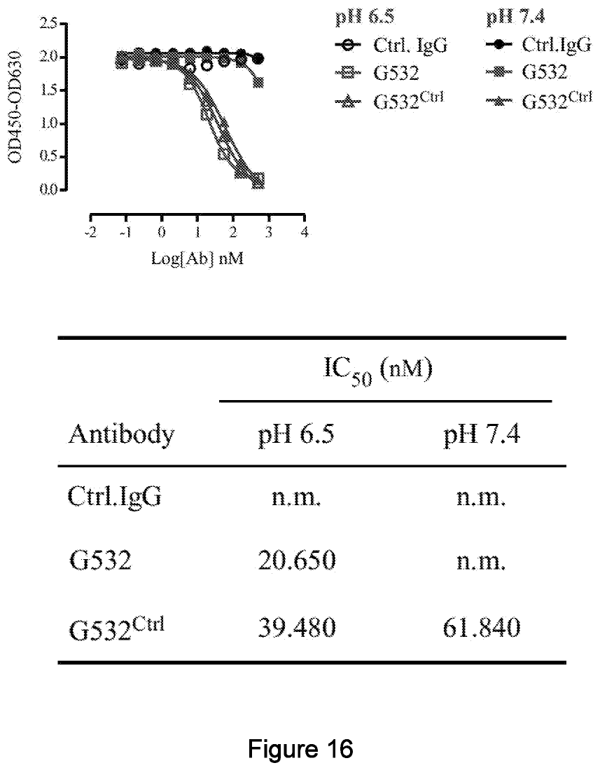

Further, the ligand-blocking ability of G532 and G532^{Ctrl} at pH 6.5 and pH7.4 was compared with ELISA in a manner similar to Example 1. As shown in FIG. 16, G532 could block EGFR-EGF binding at pH 6.5 but not at pH 7.4, in contrast, G532^{Ctrl} could block EGFR-EGF binding at both pH 6.5 and pH 7.4. This result indicates that G532 exhibits pH-dependent ligand-blocking ability.

Collectively, G532 was generated as a pH-dependent anti-EGFR antibody with potent pH-dependent ligand-blocking ability. In addition, a non-pH-dependent variant of G532^{Ctrl} was also generated and can be used for further characterization of the therapeutic utility of G532 in mice tumor models.

### Example 7. Tumor Penetration, Tumor Selectivity, and Antitumor Activity of G532

This example shows that the binding pH-dependency of G532 promotes tumor penetration, tumor selectivity, and antitumor activity.

As G532 and its variant G532^{Ctrl} exhibit strong binding cross-reactivity with human and mouse EGFR (Table 1 and FIG. 13), their binding selectivity between normal tissues and tumors in mouse models was investigated.

CB-17 SCID mice bearing A-431 tumors were treated with G532, G532^{Ctrl}, or Control IgG (10 mg/kg, in human IgG1 form) by intraperitoneal injection. 48 hours later, the mice were sacrificed to collect the A-431 tumors and mice livers for immunofluorescence staining against human IgG to detect antibody accumulation.

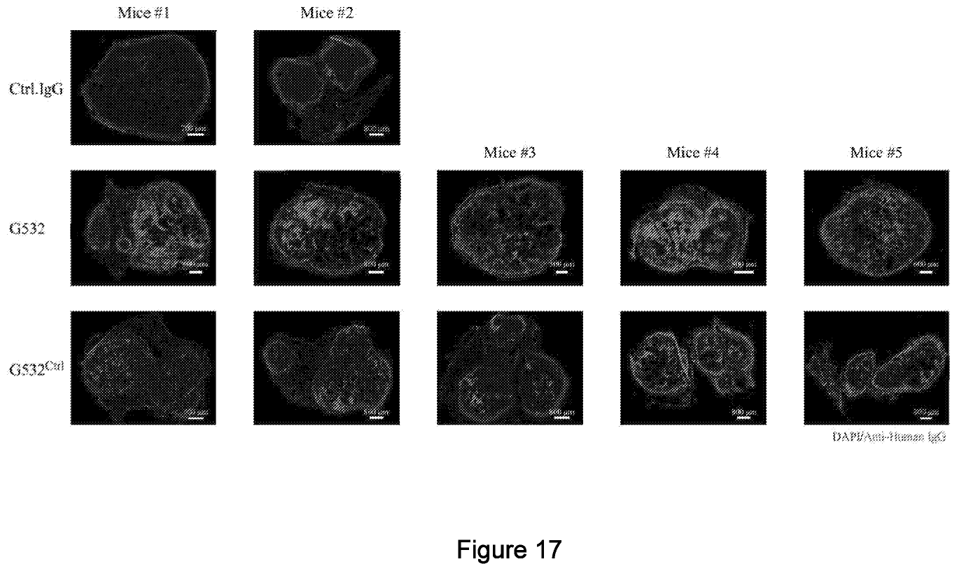

First, the distributions of G532 and G532^{Ctrl} in mouse tumors were monitored. Frozen sections of A-431 tumors were prepared, and fixed in pH 6.5 4% PFA before staining with Alexa Fluor 633-conjugated anti-human IgG secondary antibody. The penetration of indicated antibodies was then assessed based on the fluorescence intensity of Alexa Fluor 633. Results are shown in FIG. 17.

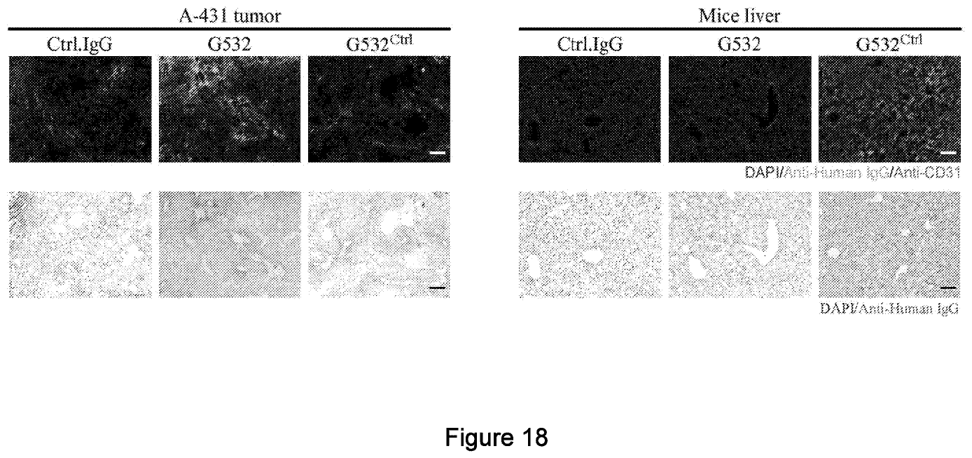

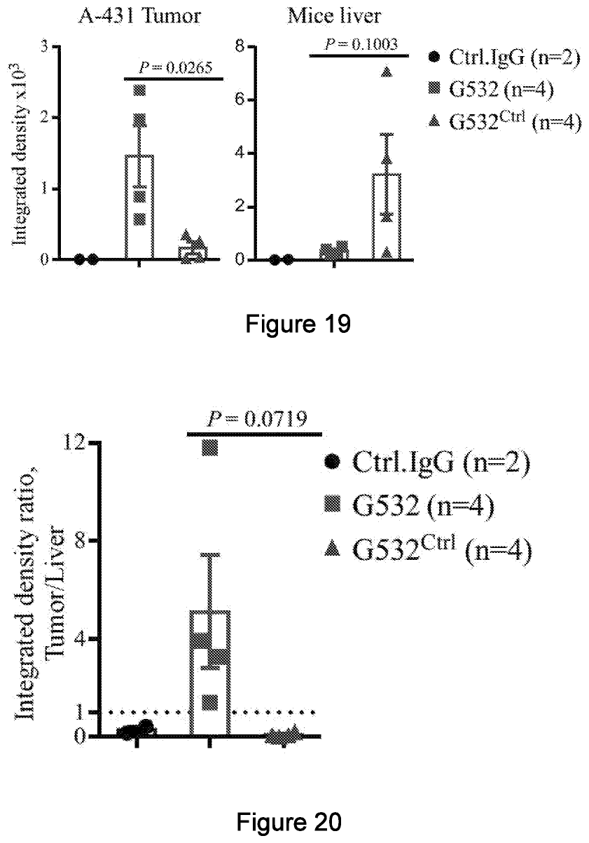

The accumulation of G532 and G532^{Ctrl} in A-431 tumors and mouse livers was analyzed by immunofluorescence staining. CB17-SCID mice bearing A-431 tumors were treated with Control IgG, G532, or G532^{Ctrl} (10 mg/kg, in human IgG1 form) by intraperitoneal injection, and then sacrificed 48 hr later to collect mice livers and A-431 tumors. Frozen sections of mice livers and A-431 tumors were prepared, and fixed in pH 6.5 4% PFA before staining. The sections were then incubated with Rat anti-mouse CD31 prior to staining with FITC-conjugated anti-human IgG secondary antibody and Alexa Fluor 647-conjugated anti-Rat IgG secondary antibody. The Alexa Fluor 647 signal indicates tumor vasculature. Antibody enrichment was assessed based on the fluorescence intensity for FITC. Images are shown in FIG. 18 and quantitative data are shown in FIG. 19 and FIG. 20.

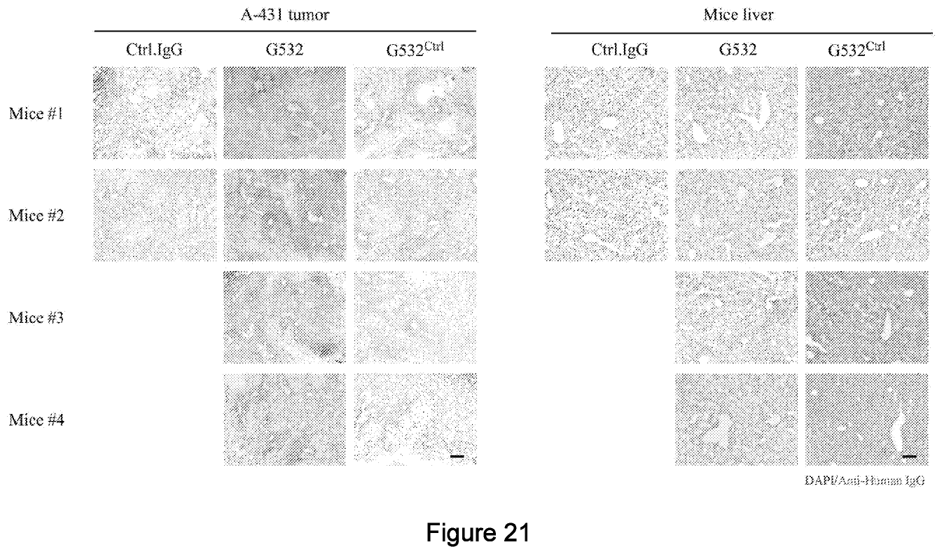

In repeated experiments, tumors from mice treated with G532 showed a stronger, and more uniform human IgG fluorescence signals than did tumors from mice treated with G532^{Ctrl} (FIGS. 17, 18, 19 and 21), indicating that G532 achieves superior tumor penetration compared to G532^{Ctrl}. Two factors may have contributed to this finding: 1) The higher binding affinity of G532^{Ctrl} over G532 at pH 7.4 would suggest that a higher proportion of the G532^{Ctrl} population would engage in binding to EGFR in normal tissues than G532. Subsequently, less G532^{Ctrl} would be available to reach tumor sites than G532 after intraperitoneal injection. 2) Previous research has shown that the pH in tumors tends to decrease with increasing distance from capillaries (Helmlinger, G. et al., (1997). Interstitial pH and pO2 gradients in solid tumors in vivo: high-resolution measurements reveal a lack of correlation. Nat. Med. 3, 177-182). Thus, G532's superior tumor penetration may relate to its increasing binding selectivity at acidic pH along with increasing distance from capillaries in tumors.

When assessing human IgG signals in livers of mice treated with G532 or G532^{Ctrl}, it was found that the average intensity of the human IgG signal in the G532 group appeared to be much lower (~9-fold) than the G532^{Ctrl} group (FIGS. 18-19 and 21). In addition, calculating the human IgG signal ratio between A-431 tumors and mice livers showed that all of the A-431 tumors in the G532 group had higher human IgG signals than mice livers (intensity ratio, tumor/liver>1); in contrast, all of the A-431 tumors in the G532^{Ctrl} group had lower human IgG signals than mice livers (intensity ratio, tumor/liver<1) (FIG. 20). These results indicate that the pH-dependency of the G532 antibody increases its tumor-selectivity relative to G532^{Ctrl}.

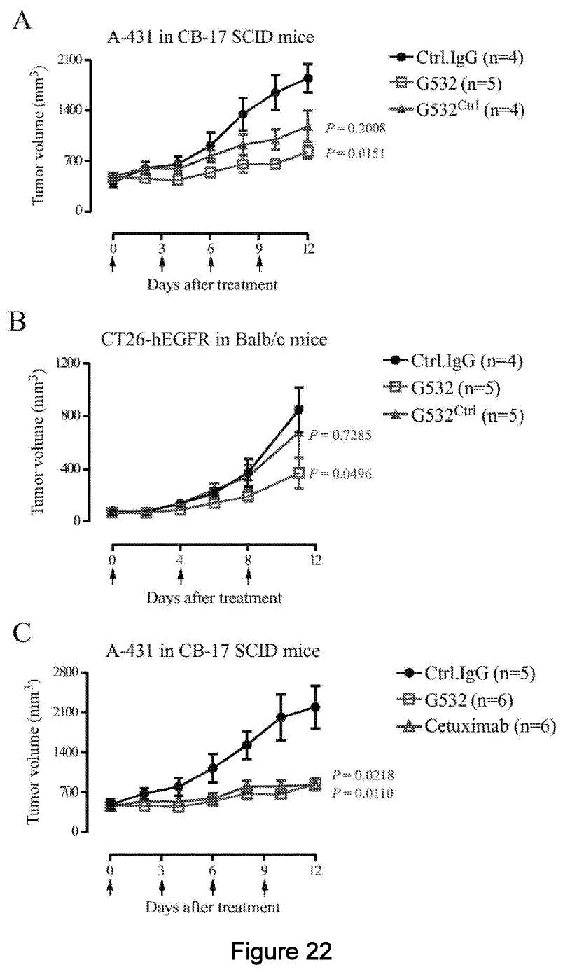

Next, the anti-tumor activity of G532 and G532^{Ctrl} was evaluated in A-431 xenograft tumor models established with CB-17 SCID mice, as well as CT26-EGFR syngeneic tumor models established with Balb/c mice. In A-431 xenograft tumor models, both G532 and G532^{Ctrl} inhibited tumor growth (FIG. 22A). However, a significant difference was only observed between the Control IgG treatment group and the G532 treatment group (FIG. 22A). In CT26-EGFR syngeneic tumor models, G532c^{Ctrl} failed to induce significant tumor growth inhibition; in contrast, G532 treatment significantly inhibited tumor growth (FIG. 22B). These results indicate that G532's superior tumor penetration increases its antitumor performance. Finally, we found that G532 elicited comparable antitumor activity with the FDA-approved therapeutic anti-EGFR mAb Cetuximab (an antibody that cannot recognize mouse EGFR) in A-431 xenograft models (FIG. 22C), supporting that G532 is an anti-EGFR antibody with clinically relevant antitumor activity.

### REFERENCES

Ogiso, H., Ishitani, R., Nureki, O., Fukai, S., Yamanaka, M., Kim, J. H., Saito, K., Sakamoto, A., Inoue, M., Shirouzu, M., et al. (2002). Crystal structure of the complex of human epidermal growth factor and receptor extracellular domains. Cell 110, 775-787.
Roskoski, R., Jr. (2014). The ErbB/HER family of protein-tyrosine kinases and cancer. Pharmacol. Res. 79, 34-74.
Cai, W. Q., Zeng, L. S., Wang, L. F., Wang, Y. Y., Cheng, J. T., Zhang, Y., Han, Z. W., Zhou, Y., Huang, S. L., Wang, X. W., et al. (2020). The Latest Battles Between EGFR Monoclonal Antibodies and Resistant Tumor Cells. Front. Oncol. 10, 1249.
Hansel, T. T., Kropshofer, H., Singer, T., Mitchell, J. A., and George, A. J. (2010). The safety and side effects of monoclonal antibodies. Nat. Rev. Drug Discov. 9, 325-338.
Achermann, Y., Frauenfelder, T., Obrist, S., Zaugg, K., Corti, N., and Gunthard, H. F. (2012). A rare but severe pulmonary side effect of cetuximab in two patients. BMJ Case Rep. 2012, bcr0320125973.
Jungbluth, A. A., Stockert, E., Huang, H. J., Collins, V. P., Coplan, K., Iversen, K., Kolb, D., Johns, T. J., Scott, A. M., Gullick, W. J., et al. (2003). A monoclonal antibody recognizing human cancers with amplification/overexpression of the human epidermal growth factor receptor. Proc. Natl. Acad. Sci. U.S.A 100, 639-644.
Johnston, R. J., Su, L. J., Pinckney, J., Critton, D., Boyer, E., Krishnakumar, A., Corbett, M., Rankin, A. L., Dibella, R., Campbell, L., et al. (2019). VISTA is an acidic pH-selective ligand for PSGL-1. Nature 574, 565-570.
Crombet Ramos, T., Mestre Fernandez, B., Mazorra Herrera, Z., and Iznaga Escobar, N. E. (2020).
Nimotuzumab for Patients With Inoperable Cancer of the Head and Neck. Front. Oncol. 10, 817.
Garrido, G., Tikhomirov, I. A., Rabasa, A., Yang, E., Gracia, E., Iznaga, N., Fernandez, L. E., Crombet, T., Kerbel, R. S., and Perez, R. (2011). Bivalent binding by intermediate affinity of nimotuzumab: a contribution to explain antibody clinical profile. Cancer Biol. Ther. 11, 373-382.
Gan, H. K., Burgess, A. W., Clayton, A. H., and Scott, A. M. (2012). Targeting of a conformationally exposed, tumor-specific epitope of EGFR as a strategy for cancer therapy. Cancer Res. 72, 2924-2930.
Hills, D., Rowlinson-Busza, G., and Gullick, W. J. (1995). Specific targeting of a mutant, activated FGF receptor found in glioblastoma using a monoclonal antibody. Int. J. Cancer 63, 537-543.
Threadgill, D. W., Dlugosz, A. A., Hansen, L. A., Tennenbaum, T., Lichti, U., Yee, D., LaMantia, C., Mourton, T., Herrup, K., Harris, R. C., et al. (1995). Targeted disruption of mouse EGF receptor: effect of genetic background on mutant phenotype. Science 269, 230-234.
Kang, J. C., Sun, W., Khare, P., Karimi, M., Wang, X., Shen, Y., Ober, R. J., and Ward, E. S. (2019). Engineering a HER2-specific antibody-drug conjugate to increase lysosomal delivery and therapeutic efficacy. Nat. Biotechnol. 37, 523-526.
Li, S., Schmitz, K. R., Jeffrey, P. D., Wiltzius, J. J., Kussie, P., and Ferguson, K. M. (2005). Structural basis for inhibition of the epidermal growth factor receptor by cetuximab. Cancer Cell 7, 301-311.

1. An isolated antibody or an antigen-binding fragment thereof that specifically binds to human EGFR (hEGFR), which comprises any one of following (a) to (h):
(a) HCDR1, HCDR2 and HCDR3 comprising or consisting of an amino acid sequence of SEQ ID NOs: 50, 61 and 52, respectively; and LCDR1, LCDR2, LCDR3 comprising or consisting of an amino acid sequence of SEQ ID NOs: 62, 15 and 54;
(b) HCDR1, HCDR2 and HCDR3 comprising or consisting of an amino acid sequence of SEQ ID NOs: 11, 12 and 13, respectively; and LCDR1, LCDR2, LCDR3 comprising or consisting of an amino acid sequence of SEQ ID NOs: 14, 15 and 16;
(c) HCDR1, HCDR2 and HCDR3 comprising or consisting of an amino acid sequence of SEQ ID NOs: 50, 51 and 52, respectively; and LCDR1, LCDR2, LCDR3 comprising or consisting of an amino acid sequence of SEQ ID NOs: 53, 15 and 54;
(d) HCDR1, HCDR2 and HCDR3 comprising or consisting of an amino acid sequence of SEQ ID NOs: 50, 61 and 52, respectively; and LCDR1, LCDR2, LCDR3 comprising or consisting of an amino acid sequence of SEQ ID NOs: 69, 70 and 54;
(e) HCDR1, HCDR2 and HCDR3 comprising or consisting of an amino acid sequence of SEQ ID NOs: 50, 61 and 52, respectively; and LCDR1, LCDR2, LCDR3 comprising or consisting of an amino acid sequence of SEQ ID NOs: 77, 70 and 54;
(f) HCDR1, HCDR2 and HCDR3 comprising or consisting of an amino acid sequence of SEQ ID NOs: 23, 24, and 25 respectively; and LCDR1, LCDR2, LCDR3 comprising or consisting of an amino acid sequence of SEQ ID NOs: 26, 27, and 28;
(g) HCDR1, HCDR2 and HCDR3 comprising or consisting of an amino acid sequence of SEQ ID NOs: 31, 32, and 33, respectively; and LCDR1, LCDR2, LCDR3 comprising or consisting of an amino acid sequence of SEQ ID NOs: 34, 35, and 36; or
(h) HCDR1, HCDR2 and HCDR3 comprising or consisting of an amino acid sequence of SEQ ID NOs: 11, 39 and 40, respectively; and LCDR1, LCDR2, LCDR3 comprising or consisting of an amino acid sequence of SEQ ID NOs: 41, 42, and 43;
wherein the hEGFR has an amino acid sequence as shown in SEQ ID NO: 1.

2. The antibody or fragment thereof according to claim 1, which comprises any one of following (a) to (h):
(a) a VH comprising or consisting of an amino acid sequence of SEQ ID NO: 55 or an amino acid sequence having at least 85% sequence identity to SEQ ID NO: 55, and a VL comprising or consisting of an amino acid sequence of SEQ ID NO: 56 or an amino acid sequence having at least 85% sequence identity to SEQ ID NO: 56;
(b) a VH comprising or consisting of an amino acid sequence of SEQ ID NO: 5 or an amino acid sequence having at least 85% sequence identity to SEQ ID NO: 5, and a VL comprising or consisting of an amino acid sequence of SEQ ID NO: 6 or an amino acid sequence having at least 85% sequence identity to SEQ ID NO: 6;
(c) a VH comprising or consisting of an amino acid sequence of SEQ ID NO: 44 or an amino acid sequence having at least 85% sequence identity to SEQ ID NO: 44, and a VL comprising or consisting of an amino acid sequence of SEQ ID NO: 45 or an amino acid sequence having at least 85% sequence identity to SEQ ID NO: 45;
(d) a VH comprising or consisting of an amino acid sequence of SEQ ID NO: 63 or an amino acid sequence having at least 85% sequence identity to SEQ ID NO: 63, and a VL comprising or consisting of an amino acid sequence of SEQ ID NO: 64 or an amino acid sequence having at least 85% sequence identity to SEQ ID NO: 64;
(e) a VH comprising or consisting of an amino acid sequence of SEQ ID NO: 71 or an amino acid sequence having at least 85% sequence identity to SEQ ID NO: 71, and a VL comprising or consisting of an amino acid sequence of SEQ ID NO: 72 or an amino acid sequence having at least 85% sequence identity to SEQ ID NO: 72;
(f) a VH comprising or consisting of an amino acid sequence of SEQ ID NO: 17 or an amino acid sequence having at least 85% sequence identity to SEQ ID NO: 17, and a VL comprising or consisting of an amino acid sequence of SEQ ID NO: 18 or an amino acid sequence having at least 85% sequence identity to SEQ ID NO: 18;
(g) a VH comprising or consisting of an amino acid sequence of SEQ ID NO: 29 or an amino acid sequence having at least 85% sequence identity to SEQ ID NO: 29, and a VL comprising or consisting of an amino acid sequence of SEQ ID NO: 30 or an amino acid sequence having at least 85% sequence identity to SEQ ID NO: 30; or
(h) a VH comprising or consisting of an amino acid sequence of SEQ ID NO: 37 or an amino acid sequence having at least 85% sequence identity to SEQ ID NO: 37, and a VL comprising or consisting of an amino acid sequence of SEQ ID NO: 38 or an amino acid sequence having at least 85% sequence identity to SEQ ID NO: 38.

3. The antibody or fragment thereof according to claim 1, which also specifically binds to mouse EGFR (mEGFR), wherein the mEGFR has an amino acid sequence as shown in SEQ ID NO: 3.

4. The antibody or fragment thereof according to claim 1, which has pH dependence in binding to hEGFR, wherein the binding activity of the antibody or fragment thereof at acidic pH is higher than the binding activity at neutral pH.

5. The antibody or fragment thereof according to claim 1, wherein the EC50 of the antibody or fragment thereof at acidic pH is at least 2-fold, 3-fold, 5-fold, 10-fold, 15-fold, 20-fold, 30-fold, 40-fold, 50-fold lower than the EC50 at neutral pH when measuring the binding of the antibody to hEGFR when the binding activity is measured by ELISA assay.

6. The antibody or fragment thereof according to claim 4, wherein the acidic pH is 5.8, 5.9, 6.0, 6.1, 6.2, 6.3, 6.4, 6.5, 6.6, 6.7 or 6.8, and the neutral pH is 7.0, 7.1, 7.2, 7.3, 7.4, 7.5 or 7.6.

7. The antibody or fragment thereof according to claim 1, wherein the antibody inhibits the binding of hEGFR to its ligand EGF.

8. The antibody or fragment thereof according to claim 1, wherein the antibody binds to one or more epitopes on hEGFR, wherein at least one epitope is different from any of the epitopes to which Cetuximab binds by at least one amino acid.

9. The antibody or fragment thereof according to claim 1, wherein the antibody binds to hEGFR via an epitope comprising histidine at animo acid position 370 (H370) and histidine at animo acid position 433 (H433) of hEGFR.

10. The antibody or fragment thereof according to claim 9, wherein the epitope comprises following amino acids on hEGFR: R377, L406, H433, Q435, and K489.

11. An isolated polynucleotide encoding the antibody or fragment thereof of claim 1.

12. An expression vector comprising the isolated polynucleotide according to claim 11.

13. A host cell comprising the expression vector according to claim 12.

14. A composition comprising the antibody or fragment thereof of claim 1 and a pharmaceutically acceptable carrier.

15. A method of reducing tumors, inhibiting the growth of tumor cells, treating a cancer or preventing recurrence of a cancer in a subject in need thereof, comprising administering to the subject a therapeutically effective amount of the antibody or fragment thereof according to claim 1.

16. The method of claim 15, wherein the tumor or cancer is EGFR-expressing tumor or cancer.

17. The method of claim 15, wherein the tumor or cancer has a microenvironment having acidic pH of 7.0, 6.9, 6.8, 6.7, 6.6, 6.5, 6.4, 6.3, 6.2, 6.1, 6.0 or lower, and the normal physiological pH of the subject is around pH 7.4.

18. The method of claim 15, wherein the tumor or cancer is selected from a group consisting of glioblastoma multiforme (GBM), head and neck cancer, and breast cancer.

19. (canceled)

20. (canceled)

21. (canceled)


SEQUENCE LISTING (77 sequences)

SEQ ID NO: 1 - protein, 1186 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 2 - protein, 618 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPK

SEQ ID NO: 3 - protein, 1186 aa, Mus musculus
LEEKKVCQGTSNRLTQLGTFEDHFLSLQRMYNNCEVVLGNLEITYVQRNYDLSFLKTIQEVAGYVLIALNTVERIPLENLQIIRGNALYENTYALAILSNYGTNRTGLRELPMRNLQEILIGAVRFSNNPILCNMDTIQWRDIVQNVFMSNMSMDLQSHPSSCPKCDPSCPNGSCWGGGEENCQKLTKIICAQQCSHRCRGRSPSDCCHNQCAAGCTGPRESDCLVCQKFQDEATCKDTCPPLMLYNPTTYQMDVNPEGKYSFGATCVKKCPRNYVVTDHGSCVRACGPDYYEVEEDGIRKCKKCDGPCRKVCNGIGIGEFKDTLSINATNIKHFKYCTAISGDLHILPVAFKGDSFTRTPPLDPRELEILKTVKEITGFLLIQAWPDNWTDLHAFENLEIIRGRTKQHGQFSLAVVGLNITSLGLRSLKEISDGDVIISGNRNLCYANTINWKKLFGTPNQKTKIMNNRAEKDCKAVNHVCNPLCSSEGCWGPEPRDCVSCQNVSRGRECVEKCNILEGEPREFVENSECIQCHPECLPQAMNITCTGRGPDNCIQCAHYIDGPHCVKTCPAGIMGENNTLVWKYADANNVCHLCHANCTYGCAGPGLQGCEVWPSGPKIPSIATGIVGGLLFIVVVALGIGLFMRRRHIVRKRTLRRLLQERELVEPLTPSGEAPNQAHLRILKETEFKKIKVLGSGAFGTVYKGLWIPEGEKVKIPVAIKELREATSPKANKEILDEAYVMASVDNPHVCRLLGICLTSTVQLITQLMPYGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASDISSILEKGERLPQPPICTIDVYMIMVKCWMIDADSRPKFRELILEFSKMARDPQRYLVIQGDERMHLPSPTDSNFYRALMDEEDMEDVVDADEYLIPQQGFFNSPSTSRTPLLSSLSATSNNSTVACINRNGSCRVKEDAFLQRYSSDPTGAVTEDNIDDAFLPVPEYVNQSVPKRPAGSVQNPVYHNQPLHPAPGRDLHYQNPHSNAVGNPEYLNTAQPTCLSSGFNSPALWIQKGSHQMSLDNPDYQQDFFPKETKPNGIFKGPTAENAEYLRVAPPSSEFIGA

SEQ ID NO: 4 - protein, 620 aa, Mus musculus
LEEKKVCQGTSNRLTQLGTFEDHFLSLQRMYNNCEVVLGNLEITYVQRNYDLSFLKTIQEVAGYVLIALNTVERIPLENLQIIRGNALYENTYALAILSNYGTNRTGLRELPMRNLQEILIGAVRFSNNPILCNMDTIQWRDIVQNVFMSNMSMDLQSHPSSCPKCDPSCPNGSCWGGGEENCQKLTKIICAQQCSHRCRGRSPSDCCHNQCAAGCTGPRESDCLVCQKFQDEATCKDTCPPLMLYNPTTYQMDVNPEGKYSFGATCVKKCPRNYVVTDHGSCVRACGPDYYEVEEDGIRKCKKCDGPCRKVCNGIGIGEFKDTLSINATNIKHFKYCTAISGDLHILPVAFKGDSFTRTPPLDPRELEILKTVKEITGFLLIQAWPDNWTDLHAFENLEIIRGRTKQHGQFSLAVVGLNITSLGLRSLKEISDGDVIISGNRNLCYANTINWKKLFGTPNQKTKIMNNRAEKDCKAVNHVCNPLCSSEGCWGPEPRDCVSCQNVSRGRECVEKCNILEGEPREFVENSECIQCHPECLPQAMNITCTGRGPDNCIQCAHYIDGPHCVKTCPAGIMGENNTLVWKYADANNVCHLCHANCTYGCAGPGLQGCEVWPSGPK

SEQ ID NO: 5 - protein, 121 aa, synthetic construct
QVQLVQSGAEVKRPGSSVKVSCKASGGTFSSYAISWVRQAPGQGLEWMGGIIPIFGTANYAQKFQGRVTITADESTSTAYMELSSLRSEDTAVYYCARDSSSGWYDYFDYWGQGTLVTVSS

SEQ ID NO: 6 - protein, 107 aa, synthetic construct
QPVLTQPPSVSAAPGAMARITCESPDIGSKSVQWYQQRPGQAPILVIYYDDDRPSGIPERFSGSNSGNTAALTINWVEPEDEADYYCQAWDDSDPVVFGGGTQLTVL

SEQ ID NO: 7 - protein, 451 aa, synthetic construct
QVQLVQSGAEVKRPGSSVKVSCKASGGTFSSYAISWVRQAPGQGLEWMGGIIPIFGTANYAQKFQGRVTITADESTSTAYMELSSLRSEDTAVYYCARDSSSGWYDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 213 aa, synthetic construct
QPVLTQPPSVSAAPGAMARITCESPDIGSKSVQWYQQRPGQAPILVIYYDDDRPSGIPERFSGSNSGNTAALTINWVEPEDEADYYCQAWDDSDPVVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - DNA, 1353 bp, synthetic construct
  source 1..1353: mol_type = other DNA
caggtgcagctggtgcagtctggggctgaggtgaagaggcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagattctagcagtggctggtacgactactttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 10 - DNA, 639 bp, synthetic construct
  source 1..639: mol_type = other DNA
cagcctgtgctgactcagccaccgtcagtgtcagcggccccaggggcgatggccagaatcacctgtgagagtcctgatattgggagtaaaagcgtgcagtggtaccagcagaggccgggccaggcccctatattagtcatctactatgatgacgaccggccctcagggatccctgagcgattctctggctccaactctgggaacacggccgccctgaccatcaactgggtcgaacccgaggatgaggccgactattattgtcaggcgtgggatgatagtgatcccgtggtcttcggcggaggcacccagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 11 - protein, 5 aa, synthetic construct
SYAIS

SEQ ID NO: 12 - protein, 17 aa, synthetic construct
GIIPIFGTANYAQKFQG

SEQ ID NO: 13 - protein, 12 aa, synthetic construct
DSSSGWYDYFDY

SEQ ID NO: 14 - protein, 11 aa, synthetic construct
ESPDIGSKSVQ

SEQ ID NO: 15 - protein, 7 aa, synthetic construct
YDDDRPS

SEQ ID NO: 16 - protein, 10 aa, synthetic construct
QAWDDSDPVV

SEQ ID NO: 17 - protein, 129 aa, synthetic construct
EVQLVQSGAEVKKPGESLMISCKGSGYSFTNHWIGWVRQMPGKGPEWMGIINPRDSDARYSPSFRGQVTFSVDRSTNTAYLQWTSLKPSDTGMYFCARRDIVVVPAATPSYYYYGMDVWGQGTTVTVSS

SEQ ID NO: 18 - protein, 107 aa, synthetic construct
DIQLTQSPSSLSASVGDSVTITCRPRQSSSRFVNWYQHKPGKAPKLLISNASDLQHGVPSRFSGRGAGTEFTLTIDSLQPDDVATYYCEQSYRFPHMFGQGTKVEIK

SEQ ID NO: 19 - protein, 459 aa, synthetic construct
EVQLVQSGAEVKKPGESLMISCKGSGYSFTNHWIGWVRQMPGKGPEWMGIINPRDSDARYSPSFRGQVTFSVDRSTNTAYLQWTSLKPSDTGMYFCARRDIVVVPAATPSYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
DIQLTQSPSSLSASVGDSVTITCRPRQSSSRFVNWYQHKPGKAPKLLISNASDLQHGVPSRFSGRGAGTEFTLTIDSLQPDDVATYYCEQSYRFPHMFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 1377 bp, synthetic construct
  source 1..1377: mol_type = other DNA
gaagtgcagctggtgcagtctggagcagaggtgaaaaagcccggggagtctctgatgatctcctgtaagggctctggatacagctttaccaaccactggattggctgggtgcgtcagatgcccgggaaaggcccggagtggatgggaatcatcaatcctcgtgactctgatgccagatacagcccgtccttccgaggccaggtcaccttttcagtcgacagatccaccaacaccgcctacctgcagtggaccagcctgaagccctcggacaccggcatgtacttctgtgcgagacgggatattgtagtagtaccagctgctaccccctcttactactactacggtatggacgtctggggccaagggaccacggtcactgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 22 - DNA, 642 bp, synthetic construct
  source 1..642: mol_type = other DNA
gacatccagttgacccagtctccatcctccctgtctgcttccgttggcgacagcgtcaccatcacttgccggccacgtcagagcagcagcagatttgttaattggtaccaacacaaaccagggaaagcccctaaactcctgatctctaatgcgtccgacttacaacatggggtgccatcgaggttcagtggcaggggagctgggacagaatttactctcaccatcgacagtctgcaacctgacgatgttgcaacctactattgtgaacagagttacagattccctcatatgtttggccaggggaccaaagtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 23 - protein, 5 aa, synthetic construct
NHWIG

SEQ ID NO: 24 - protein, 17 aa, synthetic construct
IINPRDSDARYSPSFRG

SEQ ID NO: 25 - protein, 20 aa, synthetic construct
RDIVVVPAATPSYYYYGMDV

SEQ ID NO: 26 - protein, 11 aa, synthetic construct
RPRQSSSRFVN

SEQ ID NO: 27 - protein, 7 aa, synthetic construct
NASDLQH

SEQ ID NO: 28 - protein, 9 aa, synthetic construct
EQSYRFPHM

SEQ ID NO: 29 - protein, 124 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDLEMAYYDILYGMDVWGQGTLVTVSS

SEQ ID NO: 30 - protein, 110 aa, synthetic construct
QSALTQPASVSGSAGQSITISCTGTTSDIGRYDYVSWYQQHPGQVPKLMIYDVSNRPSGVSDRFSGSKSGNTASLTISGLQTEDEADYYCSSYTTSGTRVFGGGTKLTVL

SEQ ID NO: 31 - protein, 5 aa, synthetic construct
SYYMH

SEQ ID NO: 32 - protein, 17 aa, synthetic construct
IINPSGGSTSYAQKFQG

SEQ ID NO: 33 - protein, 15 aa, synthetic construct
DLEMAYYDILYGMDV

SEQ ID NO: 34 - protein, 14 aa, synthetic construct
TGTTSDIGRYDYVS

SEQ ID NO: 35 - protein, 7 aa, synthetic construct
DVSNRPS

SEQ ID NO: 36 - protein, 10 aa, synthetic construct
SSYTTSGTRV

SEQ ID NO: 37 - protein, 128 aa, synthetic construct
EVQLVQSGAEVKKPGSSVKVSCKASGGTFSSYAISWVRQAPGQGLEWMGGIIPIFGTANYAQKFRGRVTITADESTSTAYMELSSLRSEDTAVYYCARDRDVWFGELLSYYYYGMDVWGQGTTVTVSS

SEQ ID NO: 38 - protein, 110 aa, synthetic construct
QSVVTQPPSVSAAPGQKVTISCSGSSSNIGNSYVSWYQQLPGTAPKLLIYDNNKRPSGIPDRFSGSKSGTSATLGITGLQTGDEADYYCATWDSSLSAVVFGGGTKLTVL

SEQ ID NO: 39 - protein, 17 aa, synthetic construct
GIIPIFGTANYAQKFRG

SEQ ID NO: 40 - protein, 19 aa, synthetic construct
DRDVWFGELLSYYYYGMDV

SEQ ID NO: 41 - protein, 13 aa, synthetic construct
SGSSSNIGNSYVS

SEQ ID NO: 42 - protein, 7 aa, synthetic construct
DNNKRPS

SEQ ID NO: 43 - protein, 11 aa, synthetic construct
ATWDSSLSAVV

SEQ ID NO: 44 - protein, 121 aa, synthetic construct
QVQLVQSGAEVKRPGSSVKVSCKASGGISDGSAISWVRQAPGQGLEWMGGIYFFNGNTNYAQKFQGRVTITADESTSTAYMELSSLRSEDTAVYYCARDSSLGYYDYFDYWGQGTLVTVSS

SEQ ID NO: 45 - protein, 107 aa, synthetic construct
QPVLTQPPSVSAAPGQTARITCGGNNIGEKYVQWYQQRPGQAPVLVIYYDDDRPSGIPERFSGSNSGNTAALTINSVEAGDEADYYCQAWDDYDPVVFGGGTQLTVL

SEQ ID NO: 46 - protein, 451 aa, synthetic construct
QVQLVQSGAEVKRPGSSVKVSCKASGGISDGSAISWVRQAPGQGLEWMGGIYFFNGNTNYAQKFQGRVTITADESTSTAYMELSSLRSEDTAVYYCARDSSLGYYDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 213 aa, synthetic construct
QPVLTQPPSVSAAPGQTARITCGGNNIGEKYVQWYQQRPGQAPVLVIYYDDDRPSGIPERFSGSNSGNTAALTINSVEAGDEADYYCQAWDDYDPVVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 48 - DNA, 1353 bp, synthetic construct
  source 1..1353: mol_type = other DNA
caggtgcagctggtgcagtctggggctgaggtgaagaggcctgggtcctcggtgaaggtctcctgcaaggcttctggaggcattagtgatgggtctgctatcagctgggtgcgacaggcccctggacaagggcttgagtggatgggagggatctatttttttaatggtaataccaactacgcacagaagttccagggcagagtcacgattaccgcggacgaatccacgagcacagcctacatggagctgagcagcctgagatctgaggacacggccgtgtattactgtgcgagagattcttctctgggttattacgactactttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 49 - DNA, 639 bp, synthetic construct
  source 1..639: mol_type = other DNA
cagcctgtgctgactcagccaccgtcagtgtcagcggccccagggcagaccgccagaatcacctgtggcggcaacaatattggtgagaaatacgtgcagtggtaccagcagaggccgggccaggcccctgtgttagtcatctactatgatgacgaccggccctcagggatccctgagcgattctctggctccaactctgggaacacggccgccctgaccatcaactccgtcgaagccggggatgaggccgactattattgtcaggcgtgggatgattatgatcccgtggttttcggcggaggcacccagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 50 - protein, 5 aa, synthetic construct
GSAIS

SEQ ID NO: 51 - protein, 17 aa, synthetic construct
GIYFFNGNTNYAQKFQG

SEQ ID NO: 52 - protein, 12 aa, synthetic construct
DSSLGYYDYFDY

SEQ ID NO: 53 - protein, 11 aa, synthetic construct
GGNNIGEKYVQ

SEQ ID NO: 54 - protein, 10 aa, synthetic construct
QAWDDYDPVV

SEQ ID NO: 55 - protein, 121 aa, synthetic construct
QVQLVQSGAEVKRPGSSVKVSCKASGGISDGSAISWVRQAPGQGLEWMGGIRWWGGITNYAQKFQGRVTITADESTSTAYMELSSLRSEDTAVYYCARDSSLGYYDYFDYWGQGTLVTVSS

SEQ ID NO: 56 - protein, 107 aa, synthetic construct
QPVLTQPPSVSAAPGQTARITCGGNNIGEKEVQWYQQRPGQAPVLVIYYDDDRPSGIPERFSGSNSGNTAALTINSVEAGDEADYYCQAWDDYDPVVFGGGTQLTVL

SEQ ID NO: 57 - protein, 451 aa, synthetic construct
QVQLVQSGAEVKRPGSSVKVSCKASGGISDGSAISWVRQAPGQGLEWMGGIRWWGGITNYAQKFQGRVTITADESTSTAYMELSSLRSEDTAVYYCARDSSLGYYDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 213 aa, synthetic construct
QPVLTQPPSVSAAPGQTARITCGGNNIGEKEVQWYQQRPGQAPVLVIYYDDDRPSGIPERFSGSNSGNTAALTINSVEAGDEADYYCQAWDDYDPVVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 59 - DNA, 1353 bp, synthetic construct
  source 1..1353: mol_type = other DNA
caggtgcagctggtgcagtctggggctgaggtgaagaggcctgggtcctcggtgaaggtctcctgcaaggcttctggaggcattagtgatgggtctgctatcagctgggtgcgacaggcccctggacaagggcttgagtggatgggagggatccgttggtgggggggtattaccaactacgcacagaagttccagggcagagtcacgattaccgcggacgaatccacgagcacagcctacatggagctgagcagcctgagatctgaggacacggccgtgtattactgtgcgagagattcttctctgggttattacgactactttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 60 - DNA, 639 bp, synthetic construct
  source 1..639: mol_type = other DNA
cagcctgtgctgactcagccaccgtcagtgtcagcggccccagggcagaccgccagaatcacctgtggcggcaacaatattggtgagaaagaggtgcagtggtaccagcagaggccgggccaggcccctgtgttagtcatctactatgatgacgaccggccctcagggatccctgagcgattctctggctccaactctgggaacacggccgccctgaccatcaactccgtcgaagccggggatgaggccgactattattgtcaggcgtgggatgattatgatcccgtggttttcggcggaggcacccagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 61 - protein, 17 aa, synthetic construct
GIRWWGGITNYAQKFQG

SEQ ID NO: 62 - protein, 11 aa, synthetic construct
GGNNIGEKEVQ

SEQ ID NO: 63 - protein, 121 aa, synthetic construct; identical to SEQ ID NO: 55

SEQ ID NO: 64 - protein, 107 aa, synthetic construct
QPVLTQPPSVSAAPGQTARITCGGNNIGEKHVQWYQQRPGQAPVLVIYYDSTRPSGIPERFSGSNSGNTAALTINSVEAGDEADYYCQAWDDYDPVVFGGGTQLTVL

SEQ ID NO: 65 - protein, 451 aa, synthetic construct; identical to SEQ ID NO: 57

SEQ ID NO: 66 - protein, 213 aa, synthetic construct
QPVLTQPPSVSAAPGQTARITCGGNNIGEKHVQWYQQRPGQAPVLVIYYDSTRPSGIPERFSGSNSGNTAALTINSVEAGDEADYYCQAWDDYDPVVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 67 - DNA, 1353 bp, synthetic construct; identical to SEQ ID NO: 59
  source 1..1353: mol_type = other DNA

SEQ ID NO: 68 - DNA, 639 bp, synthetic construct
  source 1..639: mol_type = other DNA
cagcctgtgctgactcagccaccgtcagtgtcagcggccccagggcagaccgccagaatcacctgtggcggcaacaatattggtgagaaacacgtgcagtggtaccagcagaggccgggccaggcccctgtgttagtcatctactatgatagcacccggccctcagggatccctgagcgattctctggctccaactctgggaacacggccgccctgaccatcaactccgtcgaagccggggatgaggccgactattattgtcaggcgtgggatgattatgatcccgtggttttcggcggaggcacccagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 69 - protein, 11 aa, synthetic construct
GGNNIGEKHVQ

SEQ ID NO: 70 - protein, 7 aa, synthetic construct
YDSTRPS

SEQ ID NO: 71 - protein, 121 aa, synthetic construct; identical to SEQ ID NO: 55

SEQ ID NO: 72 - protein, 107 aa, synthetic construct
QPVLTQPPSVSAAPGQTARITCGGNNIGENHVQWYQQRPGQAPVLVIYYDSTRPSGIPERFSGSNSGNTAALTINSVEAGDEADYYCQAWDDYDPVVFGGGTQLTVL

SEQ ID NO: 73 - protein, 451 aa, synthetic construct; identical to SEQ ID NO: 57

SEQ ID NO: 74 - protein, 213 aa, synthetic construct
QPVLTQPPSVSAAPGQTARITCGGNNIGENHVQWYQQRPGQAPVLVIYYDSTRPSGIPERFSGSNSGNTAALTINSVEAGDEADYYCQAWDDYDPVVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 75 - DNA, 1353 bp, synthetic construct; identical to SEQ ID NO: 59
  source 1..1353: mol_type = other DNA

SEQ ID NO: 76 - DNA, 639 bp, synthetic construct
  source 1..639: mol_type = other DNA
cagcctgtgctgactcagccgccgtcagtgtcagcggccccagggcagaccgccagaatcacctgtggcggcaacaatattggtgagaaccacgtgcagtggtaccagcagaggccgggccaggcccctgtgttagtcatctactatgatagcacccggccctcagggatccctgagcgattctctggctccaactctgggaacacggccgccctgaccatcaactccgtcgaagccggggatgaggccgactattattgtcaggcgtgggatgattatgatcccgtggttttcggcggaggcacccagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 77 - protein, 11 aa, synthetic construct
GGNNIGENHVQ